(19) United States
(12) Patent Application Publication
(10) Pub. No.: US 2009/0233913 A1
(43) Pub. Date: Sep. 17, 2009

(54) OPTICAL ISOMERS OF DIHYDRO-2,3-BENZODIAZEPINES AND THEIR STEREOSELECTIVE SYNTHESIS

(76) Inventors: Istvan Ling, Budapest (HU); Jozsef Barkoczy, Budapest (HU); Zoltan Greff, Budapest (HU); Gabor Szenasi, Urom (HU); Gabor Gigler, Budapest (HU); Szabolcs Kertesz, Fot (HU); Gyula Szucs, Budapest (HU); Mihaly Albert, Budapest (HU); Gabor Kapus, Pecel (HU); Geza Szabo, Budapest (HU); Miklos Vegh, Budapest (HU); Marta Agoston, Budapest (HU); Gyorgy Levay, Budakeszi (HU); Krisztina Moricz, Budapest (HU); Laszlo Gabor Harsing, Budapest (HU)

Correspondence Address: K.F. ROSS P.C., 5683 RIVERDALE AVENUE, SUITE 203 BOX 900, BRONX, NY, 10471-0900, US

(21) Appl. No.: 12/159,251

(22) PCT Filed: Dec. 29, 2006
(86) PCT No.: PCT/HU2006/000130

§ 371 Date: Dec. 22, 2008

(30) Foreign Application Priority Data
Dec. 30, 2005 (HU) P0501211
Dec. 30, 2005 (HU) P0501212

(51) Int. Cl. A61K 31/551; C07D 243/02; C07D 311/76; C07C 243/28; A61P 25/08

(52) U.S. Cl. 514/221; 540/567; 549/406; 564/151

(57) ABSTRACT

The present invention relates to dihydro-2,3-benzodiazepine compounds of high enantiomeric purity according to the general formula (I), which contain an asymmetric centre at the position 4 of the dihydro-2,3-benzodiazepine compound, and the preparation thereof and the used intermediates as well. These compounds have anti-convulsiveA muscle relaxant and neuroprotective effect due their non-competitive AMPA antagonistic properties.

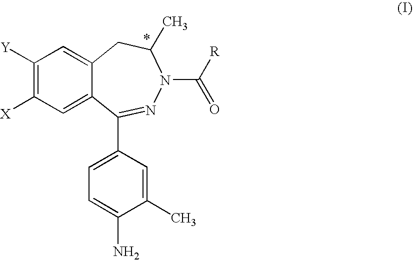

### FIELD OF THE INVENTION

The present invention relates to dihydro-2,3-benzodiazepine compounds according to the general formula

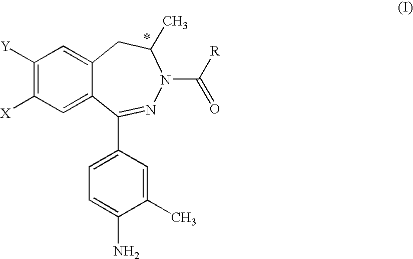

of high enantiomeric purity, wherein a methyl group and a hydrogen atom, are present at the position 4 of the dihydro2,3-benzodiazepine ring, therefore they have an asymmetric centre in this position. Furthermore, the present invention provides new intermediates of high enantiomeric purity. The compounds are non-AMPA receptor antagonists having anti-convulsive, muscle relactant and neuroprotective effects.

More particularly, the present invention concerns dihydro-2,3-benzodiazepine compounds according to the general formula (I), wherein

the configuration of the chiral carbon atom is R or S,
X represents a hydrogen, halogen or chloro atom or an alkoxy group,
Y represents a hydrogen or halogene atom, or
X and Y may represent together a methylenedioxy group,
R represents a C₁₋₄ alkyl group,
and pharmaceutically acceptable acid addition salts thereof. Furthermore, the present invention also concerns the intermediates.

### TECHNICAL BACKGROUND OF THE INVENTION

The synthesis of the racemic compounds corresponding to the enantiomeric dihydro-2,3-benzodiazepines is described in the Hungarian patent application P99 02291. The synthesis of similar racemic compounds is described also in the Hungarian patent application P00 04994.

American authors described an enantioselective synthesis for the preparation of levo-rotatory dihydro-2,3-benzodiazepine compounds having the absolute configuration R in European patent application No. EP 699 677.

According to the synthesis disclosed in the EP Application the first step is the preparation of an optically pure phenylpropanol compound having the absolute configuration S. This is prepared by the microbiological reduction of the corresponding phenylacetone derivative or through a reaction of a lithium derivative prepared from 5-bromo-benzo[1,3]dioxole with an optical active propyleneoxide. The thus prepared phenylpropanol compounds having S configuration are transformed into optical active isochromane compounds, then an oxidation step under mild circumstance and a subsequent condensation with acetic acid hydrazides results optically active hydrazone compounds.

Following a mesylation reaction, a ring closure happens under basic circumstances accompanied with the inversion of the chiral carbon atom, resulting in dihydro-2,3-benzodiazepine compounds having the absolute configuration R. Dihydro-2,3-benzodiazepine compounds having the absolute configuration S are not described in the art.

Racemic dihydro-2,3-benzodiazepine compounds and the compounds described in Hungarian patent applications No. P00 04994 and P 99 02291 are non-competitive inhibitors of AMPA receptors.

As it is known, glutamate is the most important stimulating neurotransmitter in the central nervous system. The effects of glutamate are transmitted among others by NMDA, AMPA and kainate type receptors which are connected to the ion channel.

The compounds mentioned above as non-competitive antagonists of AMPA receptors have considerable muscle relactant, neuroprotective and anti-convulsive effects and can be used in certain diseases (for example epilepsy, clinical pictures accompanied with muscle-spasticity, different neurodegenerative diseases, stroke) in which the inhibition of the AMPA/kainate receptors are useful.

From the point of view of therapeutical use it is important to develop such new active pharmaceutical ingredients, which have higher therapeutical activity and is effective in lower therapeutical dose or have considerably less therapeutical side effects than the known active ingredients.

The target of the invention was to develop new active pharmaceutical ingredients, which are more advantageous from the therapeutical point of view than the compounds known from the prior art.

This target is achieved by the preparation of dihydro-2,3-benzodiazepine compounds of high enantiomeric purity.

### SUMMARY OF THE INVENTION

The present invention relates to dihydro-2,3-benzodiazepine compounds according to the general formula (I),

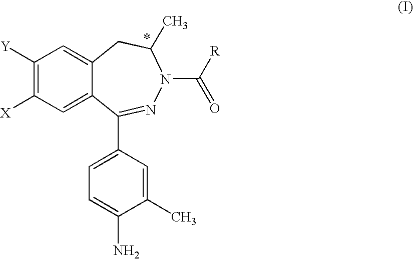

wherein the configuration of the chiral carbon atom is R or S,

X represents a hydrogen, halogen or chloro atom or alkoxy group,
Y represents a hydrogen or halogene atom, or
X and Y may represent together a methylenedioxy group,
R represents a C₁₋₄ alkyl group, preferably methyl or ethyl group,
alkoxy groups are C₁₋₄ alkoxy groups preferably a methoxy group,
and pharmaceutically acceptable acid addition salts thereof.

In the case when X and Y represent together a methylenedioxy group, the dihydro-2,3-benzodiazepines according to the general formula (I) form a 8,9-dihydro-7H-1,3-dioxolo[4,5-h][2,3]benzodiazepine ring. According to the rules of the chemical nomenclature the position of each substituents changes. This change does not influence the essence of the present invention, therefore these substituents are referred to as dihydro-2,3-benzodiazepines. However, the corresponding compounds are defined as 8,9-dihydro-7H-1,3-dioxolo[4,5-h][2,3]benzodiazepine derivatives in the Examples.

The asymmetric centres of the compounds having high purity enantiomers are marked by asterisks on the drawings, which means that the configuration of the marked carbon atoms is either R or S.

According to the present invention the expression “enantiomers of high enantiomeric purity” means practically such enantiomers which out of the possible 2 different enantiomers contain exclusively a single enantiomer or in very high concentration one enantiomer. Carrying out the invention in some cases diasteromers of high stereochemical purity are prepared. These are such diastereomers which contain at least 2 asymmetric centres and out of the 4 possible diastereomers contain exclusively one or in very high concentration only one diastereomer. Under high concentration 98% is meant.

We have found surprisingly that the levo-rotatory dihydro-2,3-benzodiazepine derivatives

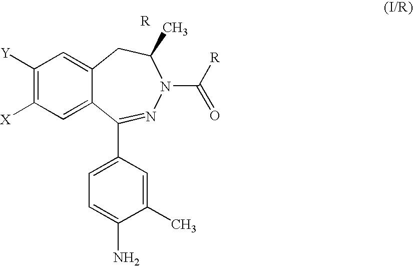

having the absolute configuration R (e.g. (R)-(−)-7-acetyl-5-(4-amino-3-methylphenyl)-8-methyl-8,9-dihydro-7H-1,3-dioxolo[4,5-h][2,3]benzodiazepine) are considerably more effective AMPA receptor antagonists than the dextro-rotatory

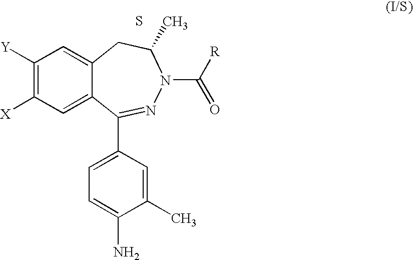

derivatives (e.g. (S)-(+)-7-acetyl-5-(4-amino-3-methylphenyl)-8-methyl-8,9-dihydro-7H-1,3-dioxolo[4,5-h][2,3]benzodiazepine) having the absolute configuration S.

Furthermore, it is very unexpected and surprising that the dextro-rotatory compounds having the absolute configuration S in case of administration per os to vigilant rats cause necrotic histological change in thymus and in bone-marrow after a one-week treatment referring to the corticosterone dominance in the thymus and in the marrow. The compounds according to the general formula (I/R) having the absolute configuration R affect the above-mentioned hystological picture only in a low degree.

Based on the above-mentioned facts the therapical use of the levo-rotatory dihydro-2,3-benzodiazepine compounds (I/R) having the absolute configuration R is advantageous, because the expected therapeutical advantages remain unchanged, meanwhile the absence of the compounds according to the general formula (I/S) having the absolute configuration S considerably reduce the probability of toxic side effects.

### DETAILED DESCRIPTION OF THE INVENTION

The above-mentioned surprising results are supported by the results of the following methods:

### “Spreading Depression” Test in the Chicken Retina

The test was carried out according to method of Sheardown (1993).

Isolated eye-cups of 5-7 days old chickens (Shaver red-brow) were used. The sexes of the chickens were unidentified. Eyes of the animals were enucleated in narcosis caused by etherization, then the back sides of the eyes were cut and put in nutrient solution.

The composition of the medium is 100 mM NaCl, 3 mM KCl, 1 mM MgSO₄, 1 mM CaCl₂, 30 mM NaHCO₃, 1 mM NaH₂PO₄ 10 mM D-glukose, pH=7.3.

The spreading depression (SD) latency generated by 5 μM S-AMPA is determined at room temperature followed by a 90-minute stabilisation period. This value was considered as control value. Then the latency caused by AMPA was measured following a 30-minute incubation period in the presence of the test compound, then followed by further 60 minutes washing the recursion of the latency time to the control value was checked.

The elongation of the control latency with 30 sec corresponds to 100% antagonism.
Literature: Sheardown, M. J.: The triggering of spreading depression in the chicken retina: a pharmacological study. Brain Research 1993, 607: 189-194.

### Toxicity Test in Rats

The examination was carried out using female Wistar rats. One day before the treatment the animals were assigned to randomised groups based on their weight (10 animals/group). Each active ingredient was suspended in a solution of 0.4% hydroxypropylmethylcellulose (Methocell F4 M, Dow Chemical Company, USA) and administered once daily for seven days by gastric-canule. The daily doses were 30 mg/kg, the animals of the control group were treated with the solvent. At the end of the experiments the animals were sacrificed by incising of the arteries of thigh in narcosis caused by ether.

After the thymus-ectomy thymi were fixed in a solution of formaline buffered with phosphate, then embedded in paraffin, pigmented with hemalaun-eosin.

Bone-marrow film preparations were made from the femur of the animals and pigmented according to Grimsa method (Sheenan D. C.-Hrapchak B. B.: Theory and practice of histotechnology (2nd ed.) Mosby Company, St. Louis, USA 1980).

The histological athrophy was scored as follows: 0—there is no change, 1—change appears, 2—slight change, 3—middle severe change, marked change, 4—severe, obvious, wide change. The rating was accomplished blind without any information about the treatment.

Groups were compared by KRUSKAL WALLIS ANOVA test (Ranks and Median). In case of significant deviation (p<0.05) WALD-WOLFOWITZ test was used for comparison.

TABLE 1
The AMPA antagonistic effect of measured compounds
in a spreading depression (SD) test, using chicken retina,
in vitro, and hystological effect in thymus and in Bone-
marrow caused by p.o. treatment of seven days on
female Wistar rats in vivo.

| | SD, EC₅₀ | thymus cortex | bone-marrow |
|---|---|---|---|
| Example | μM | atrophy (scores) | atrophy (scores) |
| 4 | 1.8 ± 0.1 | 0.30 ± 0.21 | 0.40 ± 0.27 |
| 6 | >100 | 2.9 ± 0.43** | 2.1 ± 0.31** |
| Control | — | 0 ± 0 | 0 ± 0 |
| (Solvent) | | | |

The above data show the average values and the deviation of the average values.
**= p < 0.01 vs. Example 4

According to the results shown in Table 1 above the compound of (R)-(−)-7-acetyl-5-(4-amino-3-methylphenyl)-8-methyl-8,9-dihydro-7H-1,3-dioxolo[4,5-h][2,3]benzodiazepine according to the Example 4 is an effective AMPA antagonist compound, because the effect of the AMPA receptor was blocked by 1.8 μM (EC₅₀) value in the spreading depression test, but (S)-(+)-7-acetyl-5-(4-amino-3-methylphenyl)-8-methyl-8,9-dihydro-7H-1,3-dioxolo[4,5-h][2,3]benzodiazepine according to the Example 6 has only insignificant effect on AMPA receptors, because its EC₅₀ value is higher than 100 μM. The compound of (R)-(−)-7-acetyl-5-(4-amino-3-methylphenyl)-8-methyl-8,9-dihydro-7H-1,3-dioxolo[4,5-h][2,3]benzodiazepine according to the Example 4 affected the hystological pictures of the thymus and bone-marrow in rats after a seven-day oral administration only in a minimal degree, meanwhile (S)-(+)-7-acetyl-5-(4-amino-3-methylphenyl)-8-methyl-8,9-dihydro-7H-1,3-dioxolo[4,5-h][2,3]benzodiazepine according to the Example 6 used in the same dose caused significant hystological atrophy in the thymus and the bone-marrow.

### Measurement of Plasma Corticosterone Concentration in the Rat

Examinations were carried out by using male wistar rats weighing 250-300 g. The animals were kept in an air-conditioned room, 12 hours artificial daylight was followed by 12 hours darkness. For four days prior to the experiments the animals got 0.5 ml water daily by a gastric-canule. The weight of the animals was measured at the evening before the experiments, then the animals were placed in separate cages.

Experiments were accomplished in the morning between 9 and 12 hours.

Before the treatment the animals were randomized to groups (7 animals/group)

The tested compounds were administered in a 15 mg/body weight kg dose, in 5 ml/kg solvent volume, using gastric-canule. The control animals were treated with solvent. The solvent composition contained 0.2 ml of 2.5 M HCl and 19.8 ml of distilled water.

After one hour of the treatment the animals were decapitated and about 2 ml of blood were collected in an ice-cold test tube containing 100 μl of 2% (WN) K-EDTA.

### Corticosterone Radio-Immunoassay

For the determination of corticosterone concentration, a corticosterone-3-CMO-BSA anti-body prepared in rabbit was used in a dilution of 1:40000. The antibody cross-reaction with desoxycorticosteron was 1.5%; with progesterone it was 2.3%. There were no cross-reactions with cortisolr, cortisone, cortexolone, 11-dehydrocorticosterone, 20-α-hydroxy-progesterone, 17-α-hydroxyprogesterone, 17-β oestradiol, oestrone, oestriol, testosterone and dehydroepiandrosterone. I-125 corticosterone-3-CMO-TME was used as marked compound (Izotóp Intézet, Budapest).

The corticosterone concentration was measured from 10 μl plasma without extraction. The calibration curve contained 0.027-40 pmol/test tube corticosterone. The radioactivity was measured with LKB Clinigamma apparatus.

During the statistical analysis one-aspect variance analysis and Newman-Keuls post hoc test were used.

### Examination Results:

TABLE 2
The AMPA antagonistic effect of measured compounds
in a spreading depression (SD) test, using chicken retina,
in vitro, and their effect on the plasma corticosteroid
concentration after 1 hour treatment of 15 mg/weight kg
p.o. dose in male Wistar rat, in vivo

| | | plasma corticosterone |
|---|---|---|
| | SD, EC₅₀ | concentration |
| Example | μM | pmol/ml |
| 15 | 3.9 ± 0.2 | 171.7 ± 56.5 |
| 16 | >100 | 609.9 ± 91.1*** |
| Solvent | — | 121.8 ± 29.1 |
| (Control) | | |

The data show the average values and the deviation of the average.
***= p < 0.001 vs. control.

According to the results shown in Table 2 above the (R)-(−)-3-acetyl-1-(4-amino-3-methylphenyl)-8-chloro-4-methyl-4,5-dihydro-3H-2,3-benzodiazepine according to Example 15 is an effective AMPA antagonist compound, because the effect of the AMPA receptor was blocked with 3.9 μM (EC₅₀) value in the spreading depression test, but the (S)-(+)-3-acetyl-1-(4-amino-3-methylphenyl)-8-chloro-4-methyl-4,5-dihydro-3H-2,3-benzodiazepine according to Example 16 has insignificant effect on AMPA receptors, because its EC₅₀ value is higher than 100 μM. Contrary to it, (R)-(−)-3-acetyl-1-(4-amino-3-methylphenyl)-8-chloro-4-methyl-4,5-dihydro-3H-2,3-benzodiazepine according to Example 15 did not affect the plasma corticosterone concentration after treatment per os in rats, meanwhile the (S)-(+)-3-acetyl-1-(4-amino-3-methylphenyl)-8-chloro-4-methyl-4,5-dihydro-3H-2,3-benzodiazepine according to Example 16 used in the same dose elevated the plasma corticosterone concentration.

The above-mentioned results prove that the levo-rotatory dihydro-2,3-benzodiazepine compounds according to the general formula (I/R) having the absolute configuration R (e.g. (R)-(−)-7-acetyl-5-(4-amino-3-methylphenyl)-8-methyl-8,9-dihydro-7H-1,3-dioxolo[4,5-h][2,3]benzodiazepine) or (R)-(−)-3-acetyl-1-(4-amino-3-methylphenyl)-8-chloro-4-methyl-4,5-dihydro-3H-2,3-benzodiazepine are considerably better AMPA antagonists than the dextro-rotatory compounds according to general formula (I/S) having the absolute configuration S (e.g. (S)-(+)-7-acetyl-5-(4-amino-3-methylphenyl)-8-methyl-8,9-dihydro-7H-1,3-dioxolo[4,5-h][2,3]benzodiazepine or (S)-(+)-3-acetyl-1-(4-amino-3-methylphenyl)-8-chloro-4-methyl-4,5-dihydro-3H-2,3-benzodiazepine).

The use of the dihydro-2,3-benzodiazepine compounds according to the general formula (I/R) can be advantageous for the treatment of the diseases of the central nervous system, in which the pathological activity or the pathophysical role of the glutamaterg system is proved or presumed, therefore the antagonistic effect on AMPA receptors is required.

The therapeutical use of the dihydro-2,3-benzodiazepine compounds according to the general formula (I/R) can be very advantageous for the treatment of such central nervous system disorders which require long-term administration of AMPA receptor antagonistic agents for achieving and/or maintaining the therapeutical effect.

Based on the facts above the dihydro-2,3-benzodiazepine compounds according to the general formula (I/R) can be used essentially for the treatment of stroke, traumatic brain and spinal cord injury, epilepsy, schizophrenia, central nervous tumors e.g. glioma, glioblastoma, astrocytoma, oligodendroglioma, diseases connected with muscle spasticity and neurodegenerative diseases especially Parkinson disease, Pick disease, Alzheimer disease, Huntington disease, sclerosis multiplex, Guillain-Barre syndrom, motoneuron disease (ALS), furthermore for the treatment of spasm, pain, nausea, influence on vomition, migrene, dysuria, reducing drug withdrawal symptoms or anxiety.

The dextro-rotatory compound according to the general formula (I/S) having the absolute configuration S causes significant hystological atrophy either in the thymus or in the bone-marrow of rats following a one week administration per os, meanwhile the levo-rotatory dihydro-2,3-benzodiazepine compounds according to the general formula (I/R) affect the hystological picture only in a negligible degree.

These hystological changes develop due to the long-lasting elevation of the plasma corticosterone concentration (Gopinath C.-Prentice D. E.-Lewis D. J.: Atlas of Experimental Toxicological Pathology MTP Press Limited 1987. Lancaster, England, page 124).

Based on the above-mentioned facts; the therapeutical use of the levo-rotatory dihydro-2,3-benzodiazepine compounds (I/R) having the absolute configuration R is advantageous.

The expected therapeutical advantages remain unchanged, meanwhile the absence of these compounds according to general formula (I/S) having the absolute configuration S reduces considerably the probability of toxic side effects.

More particularly, the objects of the present invention are enantiomeric dihydro-2,3-benzodiazepine derivatives according to the general formula (I), wherein

the configuration of the chiral carbon atom is R or S,
X stands for a halogen or chloro atom, preferably chloro atom,
Y stands for a halogen or chloro atom, preferably hydrogen atom, or
X and Y together may stand for a methylenedioxy group,
R stands for a C₁₋₄ alkyl group, preferably a methyl or ethyl group,
and pharmaceutically acceptable acid additional salts thereof.

Further objects of the present invention are the new intermediates having high enantiomeric purity, serving for the preparation of dihydro-2,3-benzodiazepine derivatives.

Such intermediates are dihydro-2,3-benzodiazepine derivatives of high enantiomeric purity according to the general formula

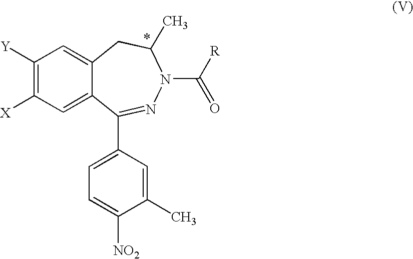

wherein
the configuration of the chiral carbon atom is R or S,
X stands for a hydrogen, halogen or chloro atom, or an alkoxy group, preferably hydrogen or chloro atom,
Y stands for a hydrogen or halogen atom, preferably hydrogen atom, or
X and Y together may stand for a methylenedioxy group,
R stands for a C₁₋₄ alkyl group, preferably a methyl or ethyl group.

Further objects of the present invention are benzo[b]pyrane derivatives according to the general formula

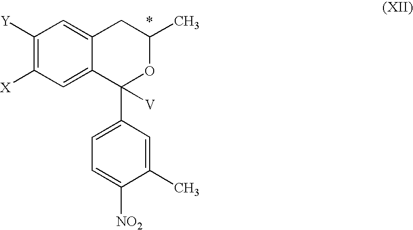

wherein
the configuration of the chiral carbon atom is R or S,
V stands for a hydrogen atom or a hydroxyl group,
X stands for a hydrogen, halogen or chloro atom, or an alkoxy group,
Y stands for a hydrogen or halogen atom, or
X and Y together may stand for methylenedioxy group.

Further objects of the present invention are hydrazone derivatives according to the general formula

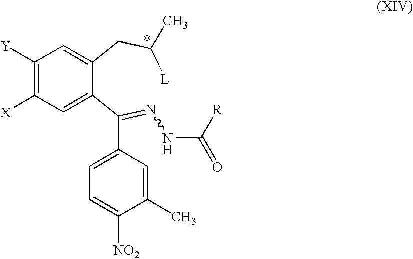

wherein the hydrazone derivative is a mixture of E and Z isomers,
the configuration of the chiral carbon atom is R or S,
L stands for a hydroxyl, alkyl or arylsulphonyl group,
X stands for a hydrogen, halogen or chloro atom or an alkoxy group,
Y stands for a halogen or halogen atom, or
X and Y together may stand for a methylenedioxy group,
R stands for a C₁₋₄ alkyl group, preferably a methyl or ethyl group.

Further objects of the present invention are racemic or enantiomeric benzodiazepine compounds according to the general formula

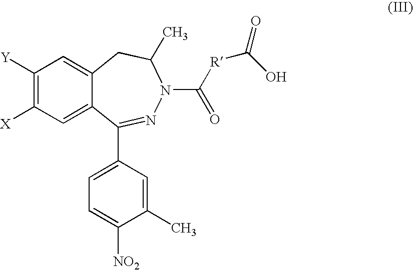

and salt formed with chiral bases thereof,
wherein
X and Y together stand for a methylenedioxy group.
R′ stands for a substituted arylene, alkylene group, preferably cis or trans alkenylene group, more preferably cis ethenylene group, and salts composed with chiral bases thereof.

Further objects of the present invention are racemic or enantiomeric benzodiazepine compounds according to the general formula

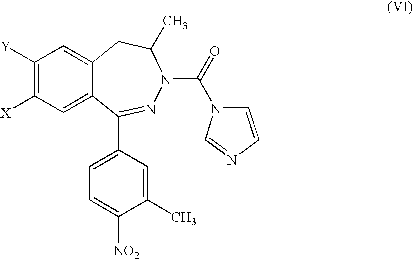

wherein
X and Y together stand for methylenedioxy group,

Enantiomeric benzodiazepine compounds are illustrated with the general formula

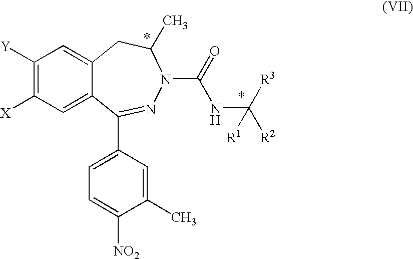

wherein
each of R¹, R², R³ is different and stands for a hydrogen atom, substituted or unsubstituted, straight or branched, saturated or unsaturated alkyl group, substituted or unsubstituted aryl or aralkyl group, R¹ preferably stands for a hydrogen atom, R² stands for a methyl group, R³ stands for a phenyl group.

Still further objects of the present invention are racemic benzodiazepine compounds according to the general formula

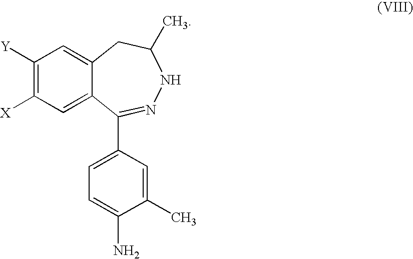

wherein
X and Y together stand for a methylenedioxy group, and acid additional salts formed with optically active acids thereof.

Further objects of the present invention are enantiomer dihydro-2,3-benzodiazepine compounds according to the general formula

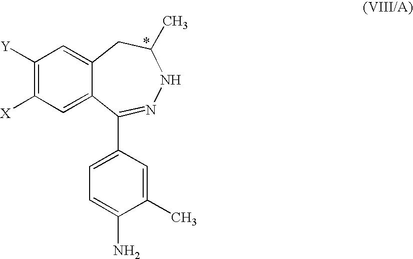

wherein
the configuration of the chiral carbon atom is R or S,
X and Y together stand for a methylenedioxy group,
and acid additional salts formed with optically active acids thereof.

Further objects of the present invention are the following groups of compounds:
(S)-(+)-5-(4-amino-3-methylphenyl)-8-methyl-7-propionyl-8,9-dihydro-7H-1,3-dioxolo[4,5-h] [2,3]benzodiazepine and pharmaceutically acceptable acid addition salts thereof,
(R)-(−)-5-(4-amino-3-methylphenyl)-8-methyl-7-propionyl-8,9-dihydro-7H-1,3-dioxolo[4,5-h] [2,3]benzodiazepine and pharmaceutically acceptable acid addition salts thereof,
(S)-(+)-7-Acetyl-5-(4-amino-3-methylphenyl)-8-methyl-8,9-dihydro-7H-1,3-dioxolo[4,5-h][2,3] benzodiazepine and pharmaceutically acceptable acid addition salts thereof,
(R)-(−)-7-Acetyl-5-(4-amino-3-methylphenyl)-8-methyl-8,9-dihydro-7H-1,3-dioxolo[4,5-h][2,3] benzodiazepine and pharmaceutically acceptable acid addition salts thereof,
(R)-(−)-3-acetyl-1-(4-amino-3-methylphenyl)-8-chloro-4-methyl-4,5-dihydro-3H-2,3-benzodiazepine and pharmaceutically acceptable acid addition salts thereof,
(S)-(+)-3-acetyl-1-(4-amino-3-methylphenyl)-8-chloro-4-methyl-4,5-dihydro-3H-2,3-benzodiazepine and pharmaceutically acceptable acid addition salts thereof,
(S)-(+)-8-methyl-5-(3-methyl-4-nitrophenyl)-7-propionyl-8,9-dihydro-7H-1,3-dioxolo[4,5-h][2,3]benzodiazepine,
(R)-(−)-8-methyl-5-(3-methyl-4-nitrophenyl)-7-propionyl-8,9-dihydro-7H-1,3-dioxolo[4,5-h][2,3]benzodiazepine,
(S)-(+)-7-acetyl-8-methyl-5-(3-methyl-4-nitrophenyl)-8,9-dihydro-7H-1,3-dioxolo[4,5-h][2,3] benzodiazepine,
(R)-(−)-7-acetyl-8-methyl-5-(3-methyl-4-nitrophenyl)-8,9-dihydro-7H-1,3-dioxolo[4,5-h][2,3] benzodiazepine,
(R)-(+)-3-acetyl-8-chloro-4-methyl-1-(3-methyl-4-nitrophenyl)-4,5-dihidro-3H-2,3-benzodiazepine,
(S)-(−)-3-acetil-8-chloro-4-methyl-1-(3-methyl-4-nitrophenyl)-4,5-dihidro-3H-2,3-benzodiazepine,
(5RS,7R)-7-Methyl-5-(3-methyl-4-nitrophenyl)-7,8-dihydro-5H-1,3-dioxolo[4,5-g]izochromane,
(5RS,7S)-7-Methyl-5-(3-methyl-4-nitrophenyl)-7,8-dihydro-5H-1,3-dioxolo[4,5-g]izochromane,
(1RS,3S)-7-chloro-3-methyl-1-(3-methyl-4-nitrophenyl)-izochromane,
(1RS,3R)-7-chloro-3-methyl-1-(3-methyl-4-nitrophenyl)-izochromane,
(5RS,7S)-7-methyl-5-(3-methyl-4-nitrophenyl)-7,8-dihydro-5H-1,3-dioxolo[4,5-g]izochroman-5-ol,
(5RS,7R)-7-methyl-5-(3-methyl-4-nitrophenyl)-7,8-dihydro-5H-1,3-dioxolo[4,5-g]izochroman-5-ol,
(1RS,3S)-7-chloro-3-methyl-1-(3-methyl-4-nitrophenyl)-izochroman-1-ol,
(1RS,3R)-7-chloro-3-methyl-1-(3-methyl-4-nitrophenyl)-izochroman-1-ol,
(S)-Acetic acid-[[6-(2-hydroxypropyl)-1,3-benzodioxol-5-yl](3-methyl-4-nitrophenyl)-methylene]hydrazide,
(S)-Acetic acid-[[6-[2-[(methylsulphonyl)-oxy]-propyl)-1,3-benzodioxol-5-yl](3-methyl-4-nitrophenyl)methylene]-hydrazide,
(S)-Propionic acid-[[6-(2-hydroxypropyl)-1,3-benzodioxol-5-yl](3-methyl-4-nitrophenyl)-methylene]hydrazide,
(S)-Propionic acid-[[6-[2-[(methylsulphonyl)-oxy]-propyl)-1,3-benzodioxol-5-yl](3-methyl-4-nitrophenyl)-methylene]-hydrazide,
(S) acetic acid [[5-chloro-2-(2-hydroxypropyl)-phenyl]-(3-methyl-4-nitro-phenyl)-methylene]-hydrazide,
(R)-Acetic acid-[[6-(2-hydroxypropyl)-1,3-benzodioxol-5-yl](3-methyl-4-nitrophenyl)-methylene]hydrazide,
(R)-Acetic acid-[[6-[2-[(methylsulphonyl)-oxy]-propyl)-1,3-benzodioxol-5-yl](3-methyl-4-nitrophenyl)-methylene]-hydrazide,
(R)-Propionic acid-[[6-(2-hydroxypropyl)-1,3-benzodioxol-5-yl](3-methyl-4-nitrophenyl)-methylene]hydrazide,
(R) acetic acid [[5-chloro-2-(2-hydroxypropyl)-phenyl]-(3-methyl-4-nitrophenyl)-methylene]hydrazide,
(R)-Propionic acid-[[6-[2-[(methylsulphonyl)-oxy]-propyl)-1,3-benzodioxol-5-yl](3-methyl-4-nitrophenyl)-methylene]-hydrazide,
(S) acetic acid [[2-[2-[(methylsulphonyl)-oxi]-propyl)-5-chlorophenyl](3-methyl-4-nitrophenyl)-methylene]hydrazide,
(R) acetic acid [[2-[2-[(methylsulphonyl)-oxi]-propyl)-5-chlorophenyl](3-methyl-4-nitrophenyl)-methylene]hydrazide
(S)-(−)- and (R)-(+)-8-Methyl-5-(3-methyl-4-nitrophenyl)-8,9-dihydro-7H-1,3-dioxolo[4,5-h][2,3] benzodiazepine,
(±)-, (S)-(+)- and (R)-(−)-4-(8-Methyl-5-(3-methyl-4-nitro-phenyl)-8,9-dihydro-7H-1,3-dioxolo[4,5-h][2,3]benzodiazepine-7-yl)-4-oxo-but-2-ene carboxylic acid,
(S)-(+)-4-(8-Methyl-5-(3-methyl-4-nitrophenyl)-8,9-dihydro-7H-1,3-dioxolo[4,5-h] [2,3] benzodiazepine-7-yl)-4-oxo-but-2-ene carboxylic acid (R)-(+)-α-methyl-benzylammonium salt,
(R)-(−)-4-(8-Methyl-5-(3-methyl-4-nitrophenyl)-8,9-dihydro-7H-1,3-dioxolo[4,5-h] [2,3] benzodiazepine-7-yl)-4-oxo-but-2-ene carboxylic acid (S)-(−)-α-methyl-benzylammonium salt,
(±)-8-Methyl-5-(3-methyl-4-nitrophenyl)-8,9-dihydro-7H-1,3-dioxolo[4,5-h][2,3] benzodiazepine-7-carboxylic acid imidazolide,
(+)-7-(N-(1(R)-phenylethyl)-carbamoyl)-8(R)-methyl-5-(3-methyl-4-nitrophenyl)-8,9-dihydro-7H-1,3-dioxolo[4,5-h][2,3]benzodiazepine,
(−)-7-(N-(1(S)-phenylethyl)-carbamoyl)-8(S)-methyl-5-(3-methyl-4-nitrophenyl)-8,9-dihydro-7H-1,3-dioxolo[4,5-h][2,3]benzodiazepine,
(±)-5-(4-amino-3-methylphenyl)-8-methyl-8,9-dihydro-7H-1,3-dioxolo[4,5-h] [2,3]benzodiazepine,
(S)-(−)-5-(4-amino-3-methylphenyl)-8-methyl-8,9-dihydro-7H-1,3-dioxolo[4,5-h][2,3] benzodiazepine, and salts formed with optically active carboxylic acids thereof,
(R)-(−)-5-(4-amino-3-methylphenyl)-8-methyl-8,9-dihydro-7H-1,3-dioxolo[4,5-h] [2,3] benzodiazepine and salts formed with optically active carboxylic acids thereof.

A still further object of the present invention is a pharmaceutical composition containing a dihydro-2,3-benzodiazepine derivative according to the general formula (I) as active ingredient or pharmaceutically acceptable acid addition salts thereof, wherein

the configuration of the chiral carbon atom is R or S,
X stands for a hydrogen, halogen, chloro atom or an alkoxy group, preferably C₁-C₄ alkoxy group, such as methoxy,
Y stands for a halogen or chloro atom, preferably hydrogen atom, or
X and Y together may stand for a methylenedioxy group,
R stands for a C₁₋₄ alkyl group, preferably methyl or ethyl group,
or in admixture with pharmaceutically acceptable vehicles.

According to the most advantageous embodiment of the present invention the active ingredient is (R)-(−)-7-acetyl-5-(4-amino-3-methylphenyl)-8-methyl-8,9-dihydro-7H-1,3-dioxolo[4,5-h][2,3]benzodiazepine or (R)-(−)-3-acetyl-1-(4-amino-3-methylphenyl)-8-chloro-4-methyl-4,5-dihydro-3H-2,3-benzodiazepine or pharmaceutically acceptable acid addition salts thereof.

The pharmaceutical compositions according to the present invention contain 0.1-95 weight %, preferably 1-50 weight %, more preferably 5-30 weight % of the active ingredient.

The pharmaceutical composition can be administered by oral, parenteral, rectal, transdermal or topical route. The dosage form of the composition can be solid or fluid.

Orally administered solid dosage forms can be e.g. powders, tablets, film tablets, microcapsules, they can contain as vehicles binding agents, e.g. sorbitol, polyvinylpyrrolidone; filling agents, e.g. lactose glucose, starch, potassium phosphate; accessories, e.g. magnesium stearate, talc, polyethyleneglycol, silica; lubricants, e.g. sodium laurylsulphate.

Orally administered liquid dosage forms are e.g. solutions, suspensions or emulsions, they may contain suspending agents, e.g. gelatine, carboxymethylcellulose; emulgeators, e.g. sorbitan monooleate; solvents, e.g. water, oils, propyleneglycol, ethanol; preservatives, e.g. p-hydroxybenzoic acid methyl or propyl ester as vehicles.

Dosage forms for parenteral administration are generally the sterile solutions of the active ingredients.

The above-mentioned dosage forms are known (e.g. Remington's Pharmaceutical Sciences, 18. edition, Mack Publishing Co., Easton, USA (1990)) from the prior art.

The pharmaceutical compositions contain generally one dosage unit. The typical daily dose of dihydro-2,3-benzodiazepine compounds according to the general formula (I) or corresponding acid addition salts thereof is 0.1-1000 mg/kg body weight for an adult. The daily dose can be administered in one or more portions per day. The effective dose depends on several factors and is established by the physician.

The pharmaceutical composition is prepared by mixing the dihydro-2,3-benzodiazepine compound according to the general formula (I) or corresponding acid addition salts thereof with one or more vehicles and the thus obtained mixture is transformed into a pharmaceutical composition in a known manner.

Applicable methods are known, for example from the above-mentioned handbook (Remington's Pharmaceutical Sciences).

Further object of the present invention is a process for the preparation of dihydro-2,3-benzodiazepine compounds according to the general formula (I), wherein the configuration of the chiral carbon atom is R or S,

X stands for a hydrogen, halogen or chloro atom, preferably a halogen or chloro atom,
Y stands for a hydrogen or halogen atom, preferably hydrogen atom, or
X and Y together may stand for a methylenedioxy group,
R stands for a C₁₋₄ alkyl group, characterized by reducing the nitro group of the corresponding compound of the formula (V). The reduction is accomplished by using stannic(II)chloride, sodium dithionite or under catalytic circumstances.

Catalytic circumstances mean using catalyst such as Raney-Ni, palladium or platinum. In case of working under catalytic circumstances, hydrogen, hydrazine hydrate, formic acid, trialkylammoniumformate or alkali formates may be used as hydrogen sources.

For the preparation of the dihydro-2,3-benzodiazepine derivatives according to the general formula (I) having high enantiomer selectivity a dihydro-2,3-benzodiazepine compound of the general formula (V) having high enantiomeric purity is used. In the course of enantioselective synthesis of the 2,3-benzodiazepine of the general formula (V) a phenyl-2-propanol derivative of the general formula (X) having high enantiomeric purity,

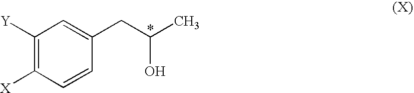

wherein X, Y and R are as defined above is reacted with a 4-nitrobenzaldehyde derivative according to general formula

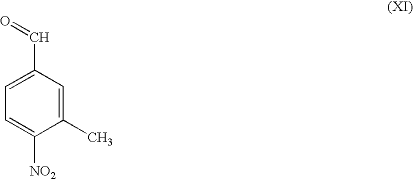

The thus obtained diastereomeric mixture of benzo[b]pyrane derivative of the general formula (XII), wherein X and Y are as defined above and V stands for a hydrogen atom, is oxidized to a hemiketal derivative of the general formula

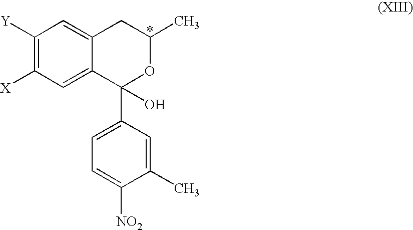

wherein X and Y are as defined above.

The diastereomeric mixture of the hemiketal compound according to the general formula (XIII) is reacted with a carboxylic acid hydrazide, preferably with acetic acid hydrazide.

The obtained hydrazone compound of the general formula

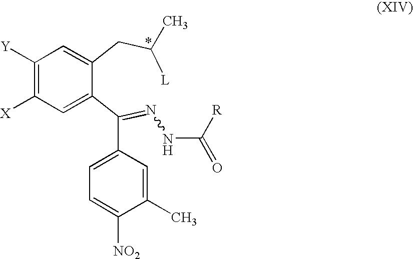

which is a mixture of E and Z isomers and wherein X, Y and R are as defined above and L stands for a hydroxyl group, is reacted with an alkylsulphonyl halogenide or an arylsulphonyl halogenide, preferably with methanesulphonyl chloride.

In course of this reaction an aryl- or alkylsulphonyl-hydrazone-type derivative is obtained according to the general formula

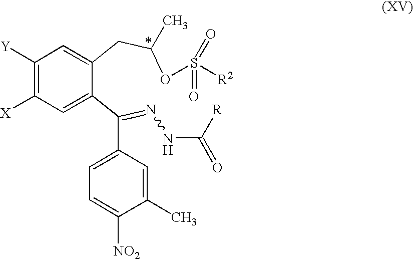

which is a mixture of E and Z isomers and wherein X, Y and R are as defined above and R² stands for an aryl, C₁₋₄ alkyl, preferably a methyl group.

This product is transformed into a dihydro-benzodiazepine derivative of the general formula (V) having high enantiomeric purity by an intramolecular cyclisation reaction, further the compound of general formula (V) is transformed into the dihydro-2,3-benzodiazepine derivative of the general formula (I) or, if necessary, into the acid addition salt thereof.

The benzo[b]pyrane derivatives according to the general formula (XII), wherein X and Y are as defined above, V stands for a hydrogen atom, are prepared by the reaction of a phenyl-2-propanol derivative according to general formula (X) of high enantiomeric purity with a 4-nitrobenzaldehyde derivative according to the general formula (XI). The reaction is carried out in an inert solvent, preferably in an aromatic hydrocarbon type solvent, more preferably in benzene or toluene between −20° C. and 150° C., preferably between 20° C. and 80° C. temperature.

The benzo[b]pyrane derivative according to the general formula (XII), wherein V stands for a hydrogen atom, X and Y are as defined above, is oxidized to the corresponding hemiketal derivative according to the general formula (XIII), wherein X and Y are as defined above. The reaction is carried out with a combination of sodium hydroxide/dimethyl sulphoxide/air in a dipolar aprotic solvent, preferably in dimethylformamide between −20° C. and 150° C., preferably between 0° C. and 50° C. temperature.

The hemiketal-type diastereomeric mixture according to the general formula (XIII) is reacted with an aliphatic carboxylic acid hydrazide, preferably acetic acid hydrazide, in aromatic or protic solvent or the mixtures thereof between −20° C. and 150° C. temperature. The reaction is accomplished preferably at the boiling point of the solvent used.

The thus obtained hydrazone-type derivative according to the general formula (XIV), which is a mixture of E and Z isomers and wherein X, Y and R are as defined above and L stands for a hydroxyl group, is reacted with an alkylsulphonyl halogenide or an arylsulphonyl halogenide compound, preferably with methanesulphonyl chloride in the presence of a tertiary amine compound, preferably triethylamine between −20° C. and 150° C. temperature in an inert solvent. As inert solvent less polar solvents, preferably chlorinated aliphatic or aromatic solvents, the most preferably dichloromethane may be used.

The obtained hydrazone-type derivative according to the general formula (VI), which is a mixture of E and Z isomers and wherein X, Y and R are as defined above and R² stands for an aryl, C₁₋₄ alkyl, preferably methyl group, is cyclized in an intramolecular cyclisation reaction by adding a base, preferably adding an alkali metal hydroxide, carbonate, hydride or alkoxyde, preferably sodium hydroxide. The cyclisation is carried out in an inert solvent, preferably in an alcohol or ether-type solvent, more preferably in methanol, ethanol, tetrahydrofurane, diethylether, diisopropylether, dioxane or mixtures thereof in a temperature range between −20° C. and 150° C. The thus obtained cyclized derivative according to the general formula (V), wherein X, Y and R are as defined above, is transformed into the dihydro-2,3-benzodiazepine derivative according to the general formula (I) of high enantiomeric purity, and if necessary the obtained products are transformed into their acid addition salts.

The cyclisation reaction, in which the sulphonate compound of the general formula (XV) is transformed into the benzodiazepine compound of the general formula (V), is accompanied by the inversion of the chirality center.

According to the most advantageous embodiment of the present invention for the preparation of (R)-(−)-7-acetyl-5-(4-amino-3-methylphenyl)-8-methyl-8,9-dihydro-7H-1,3-dioxolo[4,5-h][2,3]benzodiazepine and pharmaceutically acceptable salts thereof having high enantiomeric purity, the (S)-α-methyl-1,3-benzodioxol-5-ol of high enantiomeric purity is reacted with 3-methyl-4-nitrobenzaldehyde. The thus obtained diastereomer mixture of the benzo[b]pyrane compound according to the general formula (XII), wherein X and Y together stand for a methylenedioxy group and V stand for a hydrogen atom, is oxidized to a hemiketal-type derivative according to the general formula (XIII), wherein X and Y together stand for a methylenedioxy group.

The obtained diastereomeric mixture of hemiketal-type derivative according to the general formula (XIII) is reacted with acetic acid hydrazide. Subsequently, the obtained hydrazone type derivative, which is a mixture of E and Z isomers and wherein X and Y together stand for a methylenedioxy group and L stands for a hydroxyl group, is reacted with an alkylsulphonyl halogenide or arylsulphonyl halogenide compound, preferably with methanesulphonyl chloride. The obtained aryl or alkylsulphonylized hydrazone-type derivative of the general formula (XV), which is a mixture of E and Z isomers, and wherein R² stands for an alkyl or aryl, preferably methyl group, is cyclized by using a base, preferably alkali hydroxide, alkali carbonate, alkali hydride or alkali alcoholate, more preferably sodium hydroxide in an inert solvent, preferably in an alcohol or in an ether-type solvent, most preferably in methanol, ethanol, tetrahydrofurane, diethylether, diisopropylether, dioxane or mixtures thereof between −20° C. and 150° C. The cyclisation is accompanied by the inversion of the chirality center.

The nitro group of the obtained derivative according to the general formula (V) is reduced. The obtained dihydro-2,3-benzodiazepine derivatives according to the general formula (I) of high enantiomeric purity are transformed into a pharmaceutically acceptable acid addition salt thereof, if necessary.

An other very advantageous embodiment of the present invention is the preparation of (R)-(−)-3-acetyl-1-(4-amino-3-methylphenyl)-8-chloro-4-methyl-4,5-dihydro-3H-2,3-benzo-diazepine and pharmaceutically accepted salts thereof having high enantiomeric purity using (S)-1-(4-chlorophenyl)-propanol-2 in high enantiomeric purity according to the synthesis described above.

Dihydro-2,3-benzodiazepine derivatives according to the general formula (V) of high enantiomer purity, as intermediates for the preparation of the dihydro-2,3-benzodiazepine derivatives according to the general formula (I) of high enantiomer purity, may be prepared also as follows:

The racemic dihydro-2,3-benzodiazepine derivative according to the general formula

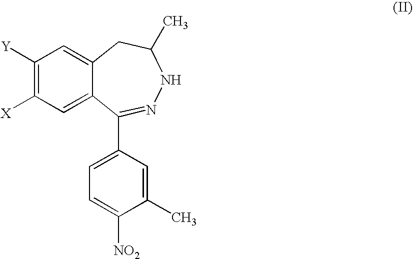

wherein X, Y and R are as defined above, is acylated with an aliphatic or aromatic dicarboxylic acid, preferably with maleic acid. The obtained racemic half amid-half acid derivative according to the general formula (III), wherein X, Y and R are as defined above, R′ stands for a substituted arylene, alkylene or alkenylene, preferably cis or trans alkenylene, most preferably cisethenylene group, is transformed into a pair of diasteromeric salts according to the general formula

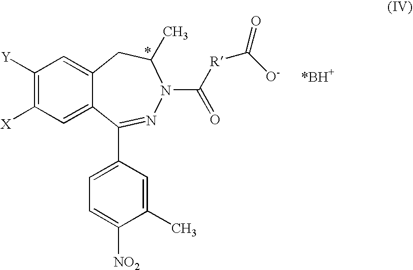

wherein *BH⁺ represents a protonated enantiomeric form of a chiral amine compound, preferably a protonated form of (S)-(−)-α-methyl-benzylamine or (R)-(+)-α-methyl-benzylamine. Thereafter the pair of diastereomeric salts is separated by a known method and the desired enantiomeric half amid-half acid derivative according to the general formula

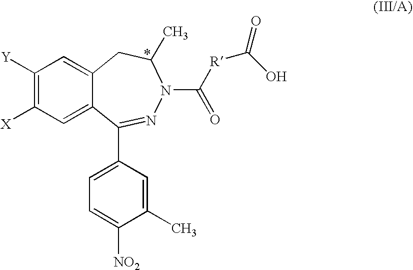

is released from its diastereomeric salt by applying a known method. Thus, the obtained enantiomeric benzodiazepine derivative according to the general formula (II/A) is acylated with an aliphatic carboxylic acid compound yielding the corresponding dihydro-2,3-benzodiazepine according to the general formula (V) having high enantiomeric purity.

For the acylation of the racemic dihydro-2,3-benzodiazepine derivative according to the general formula (II), aliphatic or aromatic dicarboxylic acid derivatives, preferably with maleic acid derivatives, most preferably with acid anhydrides can be used. The acylation can be accomplished in a known manner. According to the most preferred embodiment, the reaction is carried out in an inert solution. Preferably the racemic dihydro-2,3-benzodiazepine derivative is acylated in dichloromethane using dicarboxylic acid anhydride. The reaction can be carried out between −20° C. and 150° C., preferably between 20° C. and 80° C. temperature in the presence or absence of an organic or inorganic base. Using a base triethylamine is preferable.

Enantiomers of the obtained racemic acylated dihydro-2,3-benzodiazepine derivative according to the general formula (III) are separated with the process described above, through the diastereomer salts according to the general formula (IV), wherein the meaning of X, Y and R′ is as defined above.

It is known that the resolution of racemic salts to their enantiomers can be attained by forming a salt with an enantiomeric form of a chiral base, using appropriate solvent and circumstances. In this case the thermodynamically more stable diastereomeric salt is crystallizing from the solvent. The obtained pure diastereomeric salt containing one enantiomer of the chiral acid can be purified by recrystallisation to increase the enantiomeric purity.

Chiral bases, preferably chiral amines, e.g. (R)-(+)-α-methyl-benzylamine, (S)-(−)-α-methyl-benzylamine, (+)-dehydro-abietyl-amin, quinine, (−)-1-(4-nitrophenyl)-2-amino-1,3-propanediol or (S)-(+)-2-benzyl-amino-1-butanol can be used as a chiral base for the preparation of diastereomer salts. Most preferably (R)-(+)- or (S)-(−)-isomers of α-methyl-benzylamine can be used.

The selection of the most suitable chiral base for the preparation of diastereomeric salt depends on the appropriate dihydro-2,3-benzodiazepine derivative and the selection is a choice for those skilled in the art. The salts are prepared in dipolar aprotic solvent, preferably in ethylacetate at room temperature.

The diastereomeric salts are separated in a known manner, for example through the crystallisation of the thermodynamically more stable crystals, thereafter the obtained crystals are separated from the mother liquor. The crystals separated and enantiomerically enriched in a single enantiomer can be purified further by recrystallisation(s).

The isolated dihydro-2,3-benzodiazepine derivative containing the single enantiomer according to the general formula (IV) can be released from its diastereomeric salt by using dilute mineral acids. The obtained enantiomeric acid according to the general formula (III/A), wherein the configuration of the chiral carbon atom is R or S, is hydrolysed in the presence of lithium hydroxide and hydrogen peroxide. The reaction is carried out in an inert solvent, preferably in an ether-type solvent, most preferably in tetrahydrofurane, between −20° C. and +150° C., preferably between 20° C. and 80° C., most preferably at 50° C. temperature.

As a result of the hydrolysis enantiomeric dihydro-2,3-benzodiazepine according to the general formula (II/A)

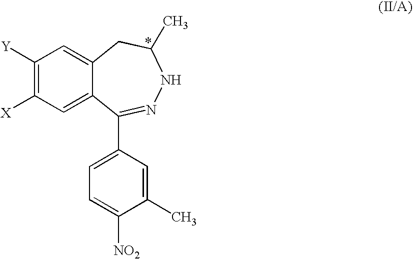

is obtained which after acylation yields the dihydro-2,3-benzodiazepine according to general formula (V).

The acylation of the dihydro-2,3-benzodiazepine derivative according to the general formula (II/A) can be carried out by using the appropriate carboxylic acid in the presence of dicyclohexylcarbodiimide. The use of acid derivatives, such as e.g. acid halogenides, preferably acid chloride compounds, or acid anhydrides is more preferred. According to the most preferable process, acetic acid anhydride or propionic acid anhydride is used.

The acylation can be carried out with or without an acid binding compound. Organic or inorganic compounds can be used as acid binding compounds. As organic acid binding compound tertiary amino-compounds, pyridine, preferably triethylamine are suitable. As inorganic acid binding agents, e.g. alkali metal or alkali earth metal carbonates or hydrogen carbonates may serve.

The acylation can be carried out in an inert solvent or without a solvent between 20° C. and 150° C. As inert solvent less polar solvents, ether-type solvents, dipolar aprotic solvents can be used. As less polar solvents, halogenated aliphatic or aromatic solvents, preferably dichloromethane, dichloroethane, chloroform or aromatic solvents or mixtures thereof, e.g. as ether-type solvent tetrahydrofurane, diethylether, diisopropylether, dioxane or mixture thereof, as dipolar aprotic solvent dimethylformamide, N-methylpyrrolidone, acetonitrile, acetone or their mixtures may serve.

According to the invention, the dihydro-2,3-benzodiazepine derivative according to the general formula (V) used as intermediate for the preparation of dihydro-2,3-benzodiazepine derivative according to the general formula (I) having high enantiomer purity, wherein X, Y and R are as defined above, may be prepared by reaction of the racemic dihydro-2,3-benzodiazepine compound according to the general formula (II), wherein X, Y and R are as defined above, with 1,1′-carbonyl-diimidazole in an inert solvent. In an advantageous embodiment this reaction is carried out in an ether-type solvent most preferably in tetrahydrofurane between −20° C. and +150° C., preferably between 20° C. and 80° C. temperature, the most preferably at the boiling point of the solvent.

The obtained racemic carbonyl-imidazolide derivative according to the general formula (VI) is reacted with a single enantiomer of a chiral amine in a dipolar aprotic solvent, preferably in dimethylformamide, N-methylpyrrolidone, acetonitrile, acetone or their mixtures, most preferably in dimethylformamide, between 20° C. and +150° C., preferably between 60° C. and 120° C. temperature.

Most preferably (R)-(+)- or (S)(−)-α-methyl-benzylamine is used as a chiral amine.

The components of the obtained diastereomeric mixture of dihydro-2,3-benzodiazepine derivatives according to the general formula (VII) are separated in the usual manner. In the formula (VII) the configuration of the chiral carbon atom one diastereomeric dihydro-2,3-benzodiazepine is R and the other is S meanwhile the configuration of the other chiral carbon atom of the diastereomeric compound is the same as that of the used chiral amine, X, Y and R are as defined above, the substituents R¹, R², R³ are different from each other, they stand for hydrogen atom, substituted or unsubstituted, straight or branched, saturated or unsaturated alkyl, substituted or unsubstituted aryl aralkyl group, preferably R¹ hydrogen atom, R² methyl group, R³ phenyl group.

The physical properties of diastereomers, such as e.g. the solubility, are considerably different. Due to the thermodynamic equilibrium in an appropriate solvent, the more stable diastereomer is precipitated from the solution, meanwhile the less stable diastereomer is staying in the solution. Appropriate solvents are alcohols, e.g. isopropanol or ethanol. The 2 diastereomers can be separated by filtration from each other, the optical purity of filtered salt can be increased by recrystallization.

The separated dihydro-2,3-benzodiazepine derivatives of high enantiomeric purity according to the general formula (VII), wherein the configuration of the one chiral carbon atom is R or S, whereas the configuration of the other chiral carbon atom depends on the used chiral amine compound, are purified optionally by recrystallisation, thereafter the diastereomers dihydro-2,3-benzodiazepine of high stereochemical purity is hydrolysed under acidic conditions, preferably between 20° C. and 80° C., most preferably at 25° C., then the obtained dihydro-2,3-benzodiazepine derivatives according to the general formula (II/A) are acylated with aliphatic carboxylic acid derivatives in a known manner resulting the dihydro-2,3-benzodiazepine derivatives of high enantiomeric purity according to the general formula (V).

The dihydro-2,3-benzodiazepine compounds according to the general formula (II/A) can be acylated as referred above.

The acylated dihydro-2,3-benzodiazepine derivatives, e.g. (R)-(−)-7-acetyl-8-methyl-5-(3-amino-4-methylphenyl)-8,9-dihydro-7H-1,3-dioxolo[4,5-h][2,3] benzodiazepine, or (R)-(−)-5-(4-amino-3-methylphenyl)-8-methyl-7-propionyl-8,9-dihydro-7H-1,3-dioxolo[4,5h][2,3] benzodiazepine, which are prepared according to any of the processes described above, are transformed into pharmaceutical acceptable salts, if required.

An object of the present invention is another process for the preparation of dihydro-2,3-benzodiazepine derivatives according to the general formula (I), wherein the configuration of the chiral carbon atom is R or S, X and Y together stand for a methylenedioxy group, R stands for a C₁₋₄ alkyl group, which comprises acylating the corresponding dihydro-2,3-benzodiazepine derivative according to general formula

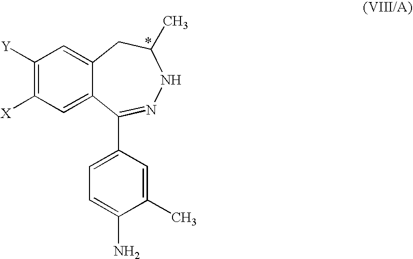

with aliphatic carboxylic acid derivatives. The acylation may be carried out with carboxylic acids using e.g. dicyclohexyl carbodiimide, or carboxylic acid derivatives, preferably acid chlorides, acid anhydrides, preferably acid anhydrides, most preferably acetic acid anhydride or propionic acid anhydride, in the presence or absence of an inert solvent, optionally in the presence of organic or inorganic acid binding agents, in a temperature range between −20° C. and 150° C.

As inorganic acid binding agent, e.g. alkali metal or alkali earth metal carbonates or hydrogen carbonates, as organic acid binding compound tertiary amine compounds, pyridine, preferably triethylamine may be selected.

Inert solvents are less polar solvents, ether-type solvents or dipolar aprotic solvents. As less polar solvents halogenated aliphatic or aromatic solvents, preferably dichloromethane, dichloroethane, chloroform or mixtures thereof may be selected. Ether-type solvents are tetrahydrofurane, diethylether, diisopropylether, dioxane or mixtures thereof. Dipolar aprotic solvents are dimethylformamide, N-methylpyrrolidone, acetonitrile, acetone or their mixtures.

In the course of the preparation of the dihydro-2,3-benzodiazepine derivative according to the general formula (VIII/A) having high enantiomer purity, wherein X, Y and R are as defined above, the racemic dihydro-2,3-benzodiazepine of the general formula (II) is reduced, then the obtained racemic dihydro-2,3-benzodiazepine derivative according to the general formula (VIII) is transformed into a pair of diastereomeric salts using a single enantiomer of an optically active organic acid, then the thus obtained pair of diastereomeric salts is separated by a known method.

Following the separation the obtained diastereomeric salt according to the general formula

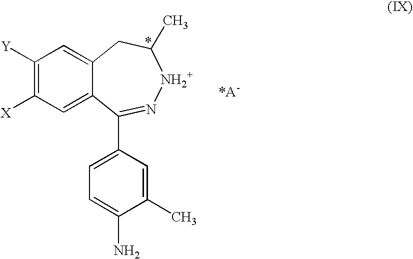

wherein *A⁻ is an anion of an optically active acid, and the configuration of the chiral carbon atom of the benzodiazepine derivative is R or S, this may be purified by recrystallisation. From the obtained dihydro-2,3-benzodiazepine derivative containing salt which is of high enantiomeric purity, the base may be released.

The reduction of the racemic dihydro-2,3-benzodiazepine derivative of the general formula (II) referred to above, is accomplished by using stanno(II)chloride, sodium dithionite or it is carried out under catalytic circumstances.

Under catalytic circumstances the catalyst used can be Raney-Ni, palladium or platinum, whereas the hydrogen source may be hydrogen, hydrazine hydrate, formic acid, trialkylammoniumformate or alkali formate.

For the preparation of diastereomeric salts an enantiomer of optically active organic acids, most preferably L- or D-tartaric acid or semi-4-chloroanilide may be used in a dipolar aprotic in an alcohol-type solvent. Dipolar aprotic solvents are acetonitrile, acetone, ethylacetate or the alcohol-type solvents are ethanol or isopropanol. The salt formation is carried out between −20° C. and 150° C., preferably between 20° C. and 80° C., most preferably at room temperature. The obtained diastereomeric salts are separated by filtration.

The filtered diastereomeric salt, which contains mainly one enantiomer of the dihydro-2,3-benzodiazepine derivative, may be purified by further recrystallisation steps. The mother liquor containing the other enantiomer compound may be evaporated and the obtained crystalline product may be recrystallised for preparing the corresponding enantiomer.

The diastereomeric salt obtained by filtration or by the recrystallisation of the residue of the evaporated mother liquor containing the single enantiomeric dihydro-2,3-benzodiazepine salt may be transformed to free single enantiomeric dihydro-2,3-benzodiazepine base by using a base. Either organic or inorganic bases, e.g. triethylamine, sodium carbonate or sodium hydrogen carbonate are suitable for this purpose.

The obtained enantiomerically pure dihydro-2,3-benzodiazepine derivatives of the general formula (VIII/A), wherein the configuration of the chiral carbon atom is R or S, are acylated as defined above to obtain the corresponding dihydro-2,3-benzodiazepine compounds according to the general formula (I) of high enantiomeric purity.

The obtained dihydro-2,3-benzodiazepine compounds, e.g. (R)-(−)-7-acetyl-8-methyl-5-(3-amino-4-methylphenyl)-8,9-dihydro-7H-1,3-dioxolo[4,5-h][2,3]benzodiazepine or (R)-(−)-5-(4-amino-3-methylphenyl)-8-methyl-7-propionyl-8,9-dihydro-7H-1,3-dioxolo[4,5-h][2,3]benzodiazepine if required may be transformed into pharmaceutical acceptable salts.

According to the invention, any pharmaceutically acceptable organic or inorganic acid may be used for the salt formation, e.g. hydrochloric acid, hydrogen bromide, sulphuric acid, phosphoric acid. Aliphatic or aromatic mono-, di-, tri- and polycarboxylic acid, further aryl or alkylsulphonic acids e.g. benzoic acid or methanesulphonic acid may be used as well. In case of using polybasic acids, preferably acidic salts such as hydrogensulphate, hemifumarate may be formed.

Still another aspect of present invention is the use of dihydro-2,3-benzodiazepine derivative according to the general formula (I) or pharmaceutically acceptable acid addition salts thereof for the preparation of pharmaceutical compositions.

These compositions are suitable for the treatment of stroke, traumatic brain and spinal cord injury, epilepsy, schizophrenia, central nervous tumors e.g. glioma, glioblastoma, astrocytoma, oligodendroglioma; diseases connected with muscle spasticity and chronic neurodegenerative diseases, especially Parkinson disease, Pick disease, Alzheimer disease, Huntington disease, sclerosis multiplex, Guillain-Barre syndrom, motoneuron disease (ALS); furthermore, for the treatment of spasm, pain, nausea, influence on vomition, migrene, dysuria, reducing drug withdrawal symptoms or anxiety.

Still further aspect of the present invention is a method of treatment of stroke, traumatic brain and spinal cord injury, epilepsy, schizophrenia, central nervous tumors e.g. glioma, glioblastoma, astrocytoma, oligodendroglioma, diseases connected with muscle spasticity and neurodegenerative diseases, especially Parkinson disease, Pick disease, Alzheimer disease, Huntington disease, sclerosis multiplex, Guillain-Barre syndrom, motoneuron disease (ALS); furthermore, the treatment of spasm, pain, nausea, influence on vomition, migrene, dysuria, reducing drug withdrawal symptoms or anxiety, by administering in a pharmaceutically effective amount to a patient in need for such treatment the compound of the dihydro-2,3-benzodiazepine derivative according to the general formula (I), wherein the configuration of the chiral carbon atom is R or S, X and Y together stand for a methylenedioxy group, R stands for a C₁₋₄ alkyl group, or a pharmaceutically acceptable acid addition salt thereof.

The new chiral dihydro-2,3-benzodiazepine derivatives according to the present invention exert a very advantageous therapeutical effect and in addition they have less side effects compared to the known active pharmaceutical ingredients, thus allowing to increase the therapeutical dose without taking into consideration the dangerous side effects of the known active ingredients.

Moreover, the present invention provides an economical process for the synthesis of the defined compounds. The aryl-2-propanol compounds of high enantiomeric purity illustrated on the general formula (X) may be prepared as described in Hungarian patent application P 04 1267 and they serve as starting substance of the stereoselective synthesis.

The present invention is shown more particularly in the examples below, without limiting the scope of the protection to the examples. The order of products and intermediates are shown below followed by the order of synthesis methods described above. The numbers of the general formula represented by a specific example is marked at the title of the examples.

### Stereoselective Synthesis

### Example 1

### (5RS,7S)-7-Methyl-5-(3-methyl-4-nitrophenyl)-7,8-dihydro-5H-1,3-dioxolo[4,5-g] izochromane (XII)

To the solution of 20.0 g (110.9 mmoles) of (S)-α-methyl-1,3-benzodioxol-5-ethanol and 18.31 g (110.9 mmoles) of 3-methyl-4-nitrobenzaldehyde in 220 ml of toluene 16.2 ml (200 mmoles) of concentrated hydrochloric acid are added. The mixture is stirred for 24 hours at room temperature. The precipitated crystals are filtered, and washed with 3×30 ml of toluene, 3×30 ml of water, then 20 ml of ethanol. The filtrate is washed with 200 ml of water, 100 ml of saturated sodium carbonate solution then with 3×100 ml of water, dried over anhydrous sodium sulphate, then evaporated. The residue is combined with the crystals which are filtered from the reaction mixture and dissolved in 400 ml of hot ethanol, then crystallised for 16 hours at room temperature. The precipitated crystals are filtered and washed with 3×30 ml of ethanol.

Thus, the yield is 21.35 g (59%) of the title product.

Melting point: 150-152° C.

[α]²⁰_{D}=+29.2° (c=1, CHCl₃)

IR (KBr): 1483, 1360, 1241, 1038. cm⁻¹

¹H-NMR (CDCl₃): 7.96 (d, J=9.0 Hz, 1H), 7.31 (m, 2H), 6.59 (s, 1H), 6.07 (s, 1H), 5.87 (d, J=1.4 Hz, 1H), 5.85 (d, J=1.4 Hz, 1H), 5.66 (s, 1H), 3.97 (m, 1H), 2.82 (dd, J1=10.9 Hz és J2=16.0 Hz, 1H), 2.68 (dd, J1=1.9 Hz és J2=16.1 Hz, 1H), 2.59 (s, 3H), 1.38 (d, J=6.1 Hz, 3H) ppm.

¹³C-NMR (CDCl₃): 148.71, 147.67, 146.54, 146.05, 134.05, 132.82, 129.40, 127.21, 127.06, 124.99, 108.31, 106.08, 100.85, 79.84, 71.40, 36.31, 21.65, 20.55. ppm.

### Example 2

### (5RS,7R)-7-Methyl-5-(3-methyl-4-nitrophenyl)-7,8-dihydro-5H-1,3-dioxolo[4,5-g]izochromane (XII)

The reaction is carried out according to Example 1 with the exception that (R)-α-methyl-1,3-benzodioxol-5-ethanol is used as starting substance.

Thus, the yield is 84% title product.

The melting point is 151-153° C.

[α]²⁰_{D}=−29.5° (c=1, CHCl₃)

IR (KBr): 1483, 1360, 1241, 1038 cm⁻¹.

¹H-NMR (CDCl₃): 7.96 (d, J=9.0 Hz, 1H), 7.31 (m, 2H), 6.59 (s, 1H), 6.07 (s, 1H), 5.87 (d, J=1.4 Hz, 1H), 5.85 (d, J=1.4 Hz, 1H), 5.66 (s, 1H), 3.97 (m, 1H), 2.82 (dd, J1=10.9 Hz és J2=16.0 Hz, 1H), 2.68 (dd, J1=1.9 Hz és J2=16.1 Hz, 1H), 2.59 (s, 3H), 1.38 (d, J=6.1 Hz, 3H) ppm.

¹³C-NMR (CDCl₃): 148.71, 147.67, 146.54, 146.05, 134.05, 132.82, 129.40, 127.21, 127.06, 124.99, 108.31, 106.08, 100.85, 79.84, 71.40, 36.31, 21.65, 20.55 ppm.

### Example 3

### (R)-(−)-7-acetyl-8-methyl-5-(3-methyl-4-nitrophenyl)-8,9-dihydro-7H-1,3-dioxolo[4,5-h][2,3] benzodiazepine (V.)

### Step A

### (5RS,7S)-7-methyl-5-(3-methyl-4-nitrophenyl)-7,8-dihydro-5H-1,3-dioxolo[4,5-g]izochroman-5-ol (XIII)

A solution of 9.82 g (30.0 mmoles) of (5RS,7S)-7-Methyl-5-(3-methyl-4-nitrophenyl)-7,8-dihydro-5H-1,3-dioxolo[4,5-g]izochromane in a mixture of 23 ml of dimethylsulphoxide and 83 ml of dimethylformamide is cooled in an ice-water bath, then 4.2 ml (42.0 mmoles) of 10 N aqueous sodium hydroxide are added. The reaction mixture is bubbled through by air for 6 hours at room temperature. The reaction mixture is then added slowly to a solution of 230 ml (230.0 mmoles) of 1N hydrochloric acid and cooled with ice-water. The precipitated product is filtered, washed with water and dried until constant weight (9.15 g). The product is a mixture of isomers and can be used in the next reaction step without further purification.

IR (KBr): 3442, 1521, 1484, 1346, 1238, 1037 cm⁻¹.

¹H-NMR (CDCl₃, 400 MHz): (major isomer) 7.92 (d, J=9.2 Hz, 1H), 7.54 (m, 2H), 6.57 (s, 1H), 6.41 (s, 1H), 6.48 (s, 1H), 5.87 (d, J=0.9 Hz, 1H), 5.85 (d, J=1.5 Hz, 1H), 4.38 (m, 1H), 3.34, (bs, 1H), 2.65-2.85 (m, 2H), 2.59 (s, 3H), 1.41 (d, J=6.2 Hz, 3H), (minor isomer) 7.98 (d, J=8.3 Hz, 1H), 7.69 (d, J=1.7 Hz, 1H), 7.68 (dd, J1=1.7 Hz, J2=7.5 Hz, 1H), 6.88 (s, 1H), 6.75 (s, 1H), 6.05 (d, J=0.8 Hz, 1H), 6.04 (d, J=0.8 Hz, 1H), 3.98 (m, 1H), 3.15, (bs, 1H), 2.65-2.85 (m, 2H), 2.63 (s, 3H), 1.42 (d, J=6.0 Hz, 3H) ppm.

### Step B

### (S)-Acetic acid-[[6-(2-hydroxypropyl)-1,3-benzodioxol-5-yl](3-methyl-4-nitrophenyl)-methylene]hydrazide (XIV)

To a solution of 3.68 g (10.7 mmoles) of (5RS,7S)-7-methyl-5-(3-methyl-4-nitrophenyl)-7,8-dihydro-5H-1,3-dioxolo[4,5-g]izochroman-5-ol (prepared in step A) in 26 ml of isopropanol, 1.03 g (13.9 mmoles) of acetic acid hydrazide and 0.22 ml (2.67 mmoles) of concentrated hydrochloric acid are added.

The solution is boiled for 6 hours, then cooled to room temperature and the solvent is evaporated under reduced pressure. The residue is dissolved in 100 ml of ethylacetate, then the obtained solution is washed with 50 ml of saturated sodium hydrogen carbonate solution, then 3×50 ml of saturated sodium chloride solution and dried over anhydrous sodium sulphate.

After the filtering off the drying agent, the solvent is evaporated in vacuo. The product is a yellow oil (3.78 g, 88%), which is a mixture of E and Z isomers in the ratio of about 1:1 and which can be used in the next reaction step without further purification.

IR (KBr): 3420, 1675, 1517, 1485, 1342, 1229, 1037 cm⁻¹.

¹H-NMR (CDCl₃, 400 MHz): 8.95 (bs, 0.5H), 8.79 (bs, 0.5H), 7.94 (d, J=8.4 Hz, 0.5H), 7.94 (d, J=9.2 Hz), 7.52 (m, 2H), 6.97 (s, 0.5H), 6.94 (s, 0.5H), 6.53 (s, 0.5H), 6.50 (s, 0.5H), 6.07 (d, J=1.2 Hz, 0.5H), 6.05 (d, J=1.2 Hz, 0.5H), 6.04 (d, J=1.2 Hz, 0.5H), 6.02 (d, J=1.2 Hz, 0.5H), 3.88 (m, 0.5H), 3.72 (m, 0.5H), 2.59 (s, 3H), 2.42 (s, 1.5H), 2.38 (s, 1.5H), 2.20-2.40 (m, 2H), 1.10 (d, J=6.1 Hz, 1.5H), 1.10 (d, J=6.1 Hz, 1.5H) ppm.

### Step C

### (S)-Acetic acid-[[6-[2-[(methylsulphonyl)-oxy]-propyl)-1,3-benzodioxol-5-yl](3-methyl-4-nitrophenyl)-methylene]hydrazide (XV)

To a solution of 3.78 g (9.5 mmoles) of (S)-acetic acid-[[6-(2-hydroxypropyl)-1,3-benzodioxol-5-yl](3-methyl-4-nitrophenyl)-methylene]hydrazide (prepared in step B) in 38 ml of dichloromethane 1.94 ml (14 mmoles) of triethylamine are added, then the reaction mixture is cooled to a temperature between 0-5° C. with ice-water. Then 0.77 ml (9.9 mmoles) of methanesulphonyl chloride is added dropwise to the reaction mixture and the reaction mixture is kept cold and stirred for additional 3 hours. The mixture is diluted with 30 ml of dichloromethane and washed with 30 ml of water, 30 ml of 1 N hydrochloric acid, then 3×30 ml of saturated sodium chloride solution, dried with sodium sulphate and the organic layer is evaporated under reduced pressure. Thus, the yield is 4.36 g of yellow oil as a mixture of optical active hydrazides, which are used in the next reaction step without further purification.

### Step D

In 90 ml of methanol, 4.36 g of (S)-acetic acid-[[6-[2-[(methylsulphonyl)-oxy]-propyl)-1,3-benzodioxol-5-yl](3-methyl-4-nitrophenyl)-methylene]hydrazide (prepared in step C) are dissolved. The solution is cooled to the temperature between 0-5° C. with ice-water and 1.0 ml (10.0 mmoles) of 10 N aqueous sodium hydroxide solution is added. The reaction mixture is stirred for 3 hours, then evaporated under reduced pressure. The residue is solidified by additional 30 ml of water, filtered and washed with 5×5 ml of water. The obtained raw product is chromatographed on silica gel using a mixture of hexane and ethylacetate. The product is recrystallized from isopropanol.

Thus 1.93 g (overall yield 30% calculated on isochromane compound) of the desired product is obtained.

The melting point of the product is 124-127° C.

[α]²⁰_{D}=−44.0° (c=1, CHCl₃)

IR (KBr): 1682, 1658, 1503, 1341, 1039 cm⁻¹.

¹H-NMR (CDCl₃): 8.00 (d, J=8.6 Hz, 1H), 7.53 (m, 2H), 6.76 (s, 1H), 6.49 (s, 1H), 6.02 (s, 2H), 5.36 (m, 1H), 3.00 (dd, J1=3.2 Hz, J2=14.6 Hz, 1H), 2.76 (dd, J1=8.5 Hz, J2=14.6 Hz, 1H), 2.64 (s, 3H), 2.29 (s, 3H), 1.08 (d, J=6.5 Hz, 3H) ppm.

¹³C-NMR: 171.84, 154.56, 149.32, 146.27, 144.19, 135.40, 133.62, 133.32, 127.66, 125.58, 124.67, 109.54, 109.51, 101.71, 58.20, 38.38, 22.83, 20.59, 18.68 ppm.

### Example 4

### (R)-(−)-7-acetyl-5-(4-amino-3-methylphenyl)-8-methyl-8,9-dihydro-7H-1,3-dioxolo[4,5-h][2,3] benzodiazepine (I/R)

In a mixture of 100 ml of dichloromethane and 10 ml of methanol 1.91 g (5.0 mmoles) of (R)-(−)-7-acetyl-8-methyl-5-(3-methyl-4-nitrophenyl)-8,9-dihydro-7H-1,3-dioxolo[4,5-h][2,3]benzodiazepine are dissolved and hydrogenated under pressure of 5,065·10⁵ Pa at room temperature in the presence of 0.10 g of 10% palladium on charcoal for 1.5 hours. The catalyst is filtered off after the hydrogenation reaction is finished, the solvent is evaporated and the raw product is recrystallized from 20 ml of methanol.

Thus 1.41 g (80%) of the desired compound is obtained.

Melting point: 123-130° C.

[α]²⁰_{D}=−479.3° (c=1, CHCl₃)

IR (KBr): 3484, 1658, 1342, 1039 cm⁻¹.

¹H-NMR (CDCl₃): 7.47 (d, J=1.3 Hz, 1H), 7.32 (dd, J1=2.1 Hz, J2=8.3 Hz, 1H), 6.77 (s, 1H), 6.65 (d, J=8.2 Hz, 1H), 6.58 (s, 1H), 6.01 (d, J=1.4 Hz, 1H), 5.97 (d, J=1.4 Hz, 1H), 5.21 (m, 1H), 3.99 (bs, 2H), 2.66 (m, 2H), 2.19 (s, 3H), 2.01 (s, 3H), 1.31 (d, J=6.3 Hz, 3H) ppm.

¹³C-NMR: 173.91, 168.63, 149.02, 147.94, 146.08, 135.19, 131.61, 129.25, 127.24, 125.97, 121.61, 113.97, 109.28, 108.63, 101.44, 61.21, 38.77, 22.53, 18.20, 17.29 ppm.

### Example 5

### (S)-(+)-7-acetyl-8-methyl-5-(3-methyl-4-nitrophenyl)-8,9-dihydro-7H-1,3-dioxolo[4,5-h][2,3] benzodiazepine (V)

The reaction is carried out according to Example 3 with the exception that (5RS,7R)-7-Methyl-5-(3-methyl-4-nitrophenyl)-7,8-dihydro-5H-1,3-dioxolo[4,5-g]izochromane (Example 2) is used as starting substance.

Thus, the yield is 30% calculated on the title product.

Melting point: 123-127° C.

[α]²⁰_{D}=+44.2° (c=1, CHCl₃)

IR (KBr): 1682, 1658, 1503, 1341, 1039 cm⁻¹.

¹H-NMR (CDCl₃): 8.00 (d, J=8.6 Hz, 1H), 7.53 (m, 2H), 6.76 (s, 1H), 6.49 (s, 1H), 6.02 (s, 2H), 5.36 (m, 1H), 3.00 (dd, J1=3.2 Hz, J2=14.6 Hz, 1H), 2.76 (dd, J1=8.5 Hz, J2=14.6 Hz, 1H), 2.64 (s, 3H), 2.29 (s, 3H), 1.08 (d, J=6.5 Hz, 3H) ppm.

¹³C-NMR: 171.84, 154.56, 149.32, 146.27, 144.19, 135.40, 133.62, 133.32, 127.66, 125.58, 124.67, 109.54, 109.51, 101.71, 58.20, 38.38, 22.83, 20.59, 18.68 ppm.

### Example 6

### (S)-(+)-7-Acetyl-5-(4-amino-3-methylphenyl)-8-methyl-8,9-dihydro-7H-1,3-dioxolo[4,5-h][2,3] benzodiazepine (I/S)

The reaction is carried out by following the molar ratios, reaction circumstances and work-up of the reaction mixture as described in Example 4 with the exception of using (S)-(+)-7-Acetyl-8-methyl-5-(3-methyl-4-nitrophenyl)-8,9-dihydro-7H-1,3-dioxolo[4,5-h][2,3]benzodiazepine as starting material (Example 5). The title product is obtained in a yield of 80%.

Melting point: 122-130° C.

[α]²⁰_{D}=+478.1° (c=1, CHCl₃)

IR (KBr): 3484, 1658, 1342, 1039 cm⁻¹.

¹H-NMR (CDCl₃): 7.47 (d, J=1.3 Hz, 1H), 7.32 (dd, J1=2.1 Hz, J2=8.3 Hz, 1H), 6.77 (s, 1H), 6.65 (d, J=8.2 Hz, 1H), 6.58 (s, 1H), 6.01 (d, J=1.4 Hz, 1H), 5.97 (d, J=1.4 Hz, 1H), 5.21 (m, 1H), 3.99 (bs, 2H), 2.66 (m, 2H), 2.19 (s, 3H), 2.01 (s, 3H), 1.31 (d, J=6.3 Hz, 3H) ppm.

¹³C-NMR: 173.91, 168.63, 149.02, 147.94, 146.08, 135.19, 131.61, 129.25, 127.24, 125.97, 121.61, 113.97, 109.28, 108.63, 101.44, 61.21, 38.77, 22.53, 18.20, 17.29 ppm.

### Example 7

### (R)-(−)-8-methyl-5-(3-methyl-4-nitrophenyl)-7-propionyl-8,9-dihydro-7H-1,3-dioxolo[4,5-h][2,3]benzodiazepine (V)

The reaction is carried out by following the molar ratios, reaction circumstances and work-up of the reaction mixture as described in Example 3 with the exception that (5RS,7S)-7-Methyl-5-(3-methyl-4-nitrophenyl)-7,8-dihydro-5H-1,3-dioxolo[4,5-g]izochromane (Example 1) is used as starting compound and propionic acid hydrazide is used as acid hydrazide compound in step B.

The product can be used without further purification.

¹H NMR (CDCl₃): 8.00 (1H, d, J=9.6 Hz), 7.54 (2H, m), 6.77 (1H, s), 6.49 (1H, s), 6.01 (2H, s), 5.37 (1H, m), 2.98 (1H, dd, J=14.5 és J=3.4 Hz), 2.76 (1H, dd, J=14.6 és J=8.7 Hz), 2.66 (2H, m), 2.64 (3H, s), 1.14 (3H, t, J=7.4 Hz), 1.09 (3H, d, J=6.5 Hz) ppm.

### Example 8

### (R)-(−)-5-(4-amino-3-methylphenyl)-8-methyl-7-propionyl-8,9-dihydro-7H-1,3-dioxolo[4,5-h] [2,3] benzodiazepine (I/R)

In a mixture of 100 ml of dichloromethane and 10 ml of methanol 1.91 g (5.0 mmoles) of (−)-8-methyl-5-(3-methyl-4-nitrophenyl)-7-propionyl-8,9-dihydro-7H-1,3-dioxolo[4,5-h][2,3]benzodiazepine are hydrogenated under the pressure of 5,065·10⁵ Pa at room temperature in the presence of 0.10 g of 10% palladium on charcoal catalyst for 1.5 hours. The catalyst is filtered off after the hydrogenation reaction is finished, the solvent is evaporated and the raw product is recrystallized from 20 ml of methanol.

Thus 1.40 g (80%) of the desired product is obtained.

Melting point: 175-177° C.

[α]²⁰_{D}=−415.4° (c=1, CHCl₃)

IR (KBr): 3355, 3245, 1631, 1038 cm⁻¹.

¹H-NMR (CDCl3, i400): 7.46 (bs, 1H), 7.33 (dd, J1=1.8 Hz, J2=8.2 Hz, 1H), 6.76 (s, 1H), 6.66 (d, J=8.3 Hz, 1H), 6.57 (s, 1H), 6.00 (d, J=1.3 Hz, 1H), 5.95 (d, J=1.3 Hz, 1H), 5.21 (m, 1H), 4.05 (b, 1H), 2.65 (m, 2H), 2.47 (m, 1H), 1.19 (m, 1H), 2.19 (s, 3H), 1.30 (d, J=6.4 Hz, 3H), 1.03 (t, J=7.5 Hz, 3H) ppm.

### Example 9

### (S)-(+)-8-methyl-5-(3-methyl-4-nitrophenyl)-7-propionyl-8,9-dihydro-7H-1,3-dioxolo[4,5-h][2,3]benzodiazepine (V)

The reaction is carried out by following the molar ratios, reaction circumstances and work-up of the reaction mixture as described in Example 3 with the exception that (5RS,7R)-7-Methyl-5-(3-methyl-4-nitrophenyl)-7,8-dihydro-5H-1,3-dioxolo[4,5-g]izochromane (Example 2) is used as starting compound and propionic acid hydrazide is used as acid hydrazide compound in step B.

The product can be used without further purification.

¹H-NMR (CDCl₃): 8.00 (1H, d, J=9.6 Hz), 7.54 (2H, m), 6.77 (1H, s), 6.49 (1H, s), 6.01 (2H, s), 5.37 (1H, m), 2.98 (1H, dd, J=14.5 és J=3.4 Hz), 2.76 (1H, dd, J=14.6 és J=8.7 Hz), 2.66 (2H, m), 2.64 (3H, s), 1.14 (3H, t, J=7.4 Hz), 1.09 (3H, d, J=6.5 Hz) ppm.

### Example 10

### (S)-(+)-5-(4-amino-3-methylphenyl)-8-methyl-7-propionyl-8,9-dihydro-7H-1,3-dioxolo[4,5-h] [2,3]benzodiazepine (I/S)

In a mixture of 100 ml of dichloromethane and 10 ml of methanol, 1.91 g (5.0 mmoles) of (S)-(+)-8-methyl-5-(3-methyl-4-nitrophenyl)-7-propionyl-8,9-dihydro-7H-1,3-dioxolo[4,5-h][2,3]benzodiazepine are hydrogenated under the pressure of 5,065·10⁵ Pa at room temperature in the presence of 0.10 g of 10% palladium on charcoal catalyst for 1.5 hours. The catalyst is filtered off after the hydrogenation reaction is finished, the solvent is evaporated and the raw product is recrystallized from 20 ml of methanol.

Thus 1.40 g (80%) of the desired product is obtained.

Melting point: 176-178° C.

[α]²⁰_{D}=+433.7° (c=1, CHCl₃)

IR (KBr): 3355, 3245, 1631, 1038 cm⁻¹.

¹H-NMR (CDCl3, i400): 7.46 (bs, 1H), 7.33 (dd, J1=1.8 Hz, J2=8.2 Hz, 1H), 6.76 (s, 1H), 6.66 (d, J=8.3 Hz, 1H), 6.57 (s, 1H), 6.00 (d, J=1.3 Hz, 1H), 5.95 (d, J=1.3 Hz, 1H), 5.21 (m, 1H), 4.05 (b, 1H), 2.65 (m, 2H), 2.47 (m, 1H), 1.19 (m, 1H), 2.19 (s, 3H), 1.30 (d, J=6.4 Hz, 3H), 1.03 (t, J=7.5 Hz, 3H) ppm.

### Example 11

### (1RS,3S)-7-Chloro-3-methyl-1-(3-methyl-4-nitrophenyl)-izochromane (XII)

To the solution of 8.9 g (52.0 mmol) of (S)-(+)-1-(4-chlorophenyl)-2-propanol and 8.58 g (52.0 mmol) of 3-methyl-4-nitrobenzaldehyde in 80 ml of anhydrous benzene 10.63 g (78.0 mmol) of powdered melting dried anhydrous zinc chloride are added, then dry hydrochloric acid gas is introduced to the reaction mixture for five hours under vigorous stirring. Then the reaction mixture is refluxed for 1.5 hours. The organic layer is decanted from the deliquescing zinc chloride layer, then the organic layer is stirred with 3×80 ml of toluene. The collected organic layers are combined and washed with 5×80 ml of 25% aqueous sodium bisulphite solution, 80 ml of saturated sodium hydrogen carbonate solution, 3×80 ml of water, dried over sodium sulphate then evaporated. The residue is dissolved in 15 ml of hot ethanol and kept for 16 hours in refrigerator. The precipitated crystals are filtered and washed with 3×5 ml of ethanol.

Thus, the yield is 3.51 g (21.2%) of the desired product. The melting point is 142-147° C.

[α]_{D}²⁰=+43.91° (c=0.5, CHCl₃), [α]₄₃₆²⁰=+120.76 (c=0.5, CHCl₃)

IR (KBr): 1518, 1342, 1075 cm⁻¹.

¹H-NMR (CDCl₃): 7.98 (m, 1H), 7.31 (m, 2H), 7.15 (dd, J1=2.1 Hz, J2=8.2 Hz, 1H), 7.08 (d, J=8.2 Hz, 1H), 6.60 (d, J=1.6 Hz, 1H), 5.70 (s, 1H), 4.00 (m, 1H), 2.85 (dd, J1=10.7 Hz, J2=16.4 Hz, 1H), 2.78 (dd, J1=2.9 Hz és J2=16.0 Hz, 1H), 2.61 (s, 3H), 1.40 (d, J=6.1 Hz, 3H) ppm.

¹³C-NMR (CDCl₃): 146.73, 138.30, 134.22, 132.90, 132.38, 131.83, 130.15, 127.28, 127.13, 126.08, 125.15, 79.59, 71.51, 35.69, 21.72, 20.60 ppm.

### Example 12

### (1RS,3R)-7-Chloro-3-methyl-1-(3-methyl-4-nitrophenyl)-izochromane (XII)

To the solution of 8.53 g (50.0 mmol) of (R)-(−)-1-(4-chlorophenyl)-2-propanol and 8.25 g (50.0 mmol) of 3-methyl-4-nitrobenzaldehyde in 80 ml of anhydrous benzene, 10.22 g (75.0 mmol) of powdered, melting dried anhydrous zinc chloride is added, then dry hydrochloric acid gas is introduced to the reaction mixture for five hours under vigorous stirring.

Then the reaction mixture is refluxed for 1.5 hours. The organic layer is decanted from the deliquescing zinc chloride layer, then the organic layer is stirred with 3×80 ml of toluene.

The collected organic layers are combined, washed with 5×80 ml of 25% aqueous sodium bisulphite solution, 80 ml of saturated sodium hydrogen carbonate solution, 3×80 ml of water, dried over sodium sulphate, then evaporated.

The residue is dissolved in 15 ml of hot ethanol and kept for 16 hours in refrigerator. The precipitated crystals are filtered and washed with 3×5 ml of ethanol.

Thus, the yield is 3.42 g (21.5%) of the desired product. The melting point is 141-144° C.

[α]_{D}²⁰=−43.19° (c=0.5, CHCl₃)

IR (KBr): 1518, 1342, 1075 cm⁻¹.

¹H-NMR (CDCl₃): 7.98 (m, 1H), 7.31 (m, 2H), 7.15 (dd, J1=2.1 Hz, J2=8.2 Hz, 1H), 7.08 (d, J=8.2 Hz, 1H), 6.60 (d, J=1.6 Hz, 1H), 5.70 (s, 1H), 4.00 (m, 1H), 2.85 (dd, J1=10.7 Hz, J2=16.4 Hz, 1H), 2.78 (dd, J1=2.9 Hz és J2=16.0 Hz, 1H), 2.61 (s, 3H), 1.40 (d, J=6.1 Hz, 3H) ppm.

¹³C-NMR (CDCl₃): 146.73, 138.30, 134.22, 132.90, 132.38, 131.83, 130.15, 127.28, 127.13, 126.08, 125.15, 79.59, 71.51, 35.69, 21.72, 20.60 ppm.

### Example 13

### (R)-(+)-3-acetyl-8-chloro-4-methyl-1-(3-methyl-4-nitrophenyl)-4,5-dihydro-3H-2,3-benzodiazepine (V)

### Step A

### (1RS,3S)-7-Chloro-3-methyl-1-(3-methyl-4-nitrophenyl)-izochroman-1-ol (XIII)

A solution of 3.35 g (10.0 mmol) of (1RS,3S)-7-chloro-3-methyl-1-(3-methyl-4-nitrophenyl)-izochromane in a mixture of 8 ml of dimethylsulphoxide and 27 ml of dimethylformamide is cooled in an ice-cold water bath, then 1.8 ml (18.0 mmol) of 10 N aqueous sodium hydroxide are added. The reaction mixture is bubbled through by air for 4 hours at room temperature.

The reaction mixture is added to a solution of 67 ml (67 mmol) of 1N hydrochloric acid cooled with ice-cold water bath. The precipitated product is filtered, washed with water and dried to constant weight (3.65 g).

The product is a mixture of isomers and can be used in the next reaction step without further purification.

IR (KBr): 3333, 1653, 1520, 1347, 1172, 1066 cm⁻¹.

¹H-NMR (CDCl₃, 500 MHz): 7.96 (d, J=9.2 Hz, 1H), 7.56 (m, 2H), 7.18 (dd, J1=2.2 Hz, J2=8.3 Hz, 1H), 7.09 (d, J=8.2 Hz, 1H), 6.96 (d, J=2.2 Hz, 1H), 4.45 (m, 1H), 2.80 (m, 2H), 2.61 (s, 3H), 1.43 (d, J=6.2 Hz, 3H) ppm.

¹³C-NMR (CDCl₃): 149.38, 138.49, 133.72, 132.33, 132.18, 130.55, 129.97, 128.98, 128.47, 127.88, 124.84, 124.79, 97.26, 65.70, 35.55, 21.30, 20.70 ppm.

### Step B

### (S) acetic acid [[5-chloro-2-(2-hydroxy-propyl)-phenyl]-(3-methyl-4-nitrophenyl)-methylene]-hydrazide (XIV)

To a solution of 3.52 g (10.0 mmol) of (1RS,3S)-7-Chloro-3-methyl-1-(3-methyl-4-nitrophenyl)-izochroman-1-ol prepared in step A, 1.16 g (15.7 mmol) of acetic acid hydrazide and 0.23 ml (2.8 mmol) of concentrated hydrochloric acid is added in 15 ml of isopropanol.

The solution is boiled for 6 hours, then cooled to room temperature and the solvent is evaporated in vacuo. The residue is dissolved in 100 ml of ethylacetate, the obtained solution is washed with 50 ml of saturated sodium hydrogen carbonate solution, 3×50 of saturated sodium chloride solution and dried over anhydrous sodium sulphate. Following the filtering off the drying agent, the solvent is evaporated in vacuo. The product is a yellow oil (3.66 g, 94%), a mixture of E and Z isomers in the ratio of about 1:1. The mixture may be used in the next reaction step without further purification.

IR (KBr): 3423, 1670, 1518, 1339, 732 cm⁻¹.

¹H-NMR (CDCl₃, 500 MHz): 7.95 (m, 1H), 7.46 (m, 4H), 7.15 (m, 0.5H), 7.09 (m, 0.5H), 3.93 (m, 0.5H), 3.71 (m, 0.5H), 5.59 (s, 3H), 2.42 (s, 1.5H), 2.39 (m, 2H), 2.35 (s, 1.5H), 1.14 (d, J=6.1 Hz, 1.5H), 1.13 (d, J=6.2 Hz, 1.5H) ppm.

### Step C

### (S) acetic acid [[2-[2-[(methylsulfonyl)-oxi]-propil)-5-chlorophenyl](3-methyl-4-nitrophenyl)-methylene]hydrazide (XV)

To a solution of 3.66 g (9.38 mmoles) of (S) acetic acid [[5-chloro-2-(2-hydroxypropyl)-phenyl]-(3-methyl-4-nitrophenyl)-methylene]-hydrazide prepared according to step B in 30 ml of dichloromethane 1.93 ml (15 mmol) of triethylamine are added, then the reaction mixture is cooled between 0-5° C. To the reaction mixture 0.86 ml (11.0 mmoles) of methanesulphonyl chloride are added dropwise and the reaction mixture is kept cold and stirred for additional 4 hours. Then the mixture is diluted with 30 ml of dichloromethane and washed with 25 ml of water, 25 ml of 1 n hydrochloric acid, then 3×25 ml of saturated sodium chloride solution, dried over sodium sulphate, then evaporated in vacuum solution. Thus, the yield is 4.14 g (94%) of yellow oil, a mixture of optical active hydrazides, which are used in the next reaction step without further purification.

IR (KBr): 3181, 1683, 1520, 1330, 1172 cm⁻¹.

¹H-NMR (CDCl₃, 500 MHz): 8.33 (bs, 0.4*1H), 8.30 (bs, 0.6*1H), 7.38-7.56 (m, 4H), 7.16 (d, J=2.2 Hz, 0.4*1H), 7.14 (d, J=2.2 Hz, 0.6*1H), 4.83 (m, 1H), 2.90 (s, 0.6*3H), 2.83 (s, 0.4*1H), 2.65 (m, 2H), 2.60 (s, 0.4*3H), 2.59 (s, 0.6*3H), 2.48 (s, 0.6*3H), 2.47 (s, 0.4*3H), 1.32 (d, J=6.5 Hz, 0.4*3H), 1.31 (d, J=6.3 Hz, 0.4*3H) ppm.

### Step D

### (R)-(+)-3-acetil-8-chloro-4-methyl-1-(3-methyl-4-nitrophenyl)-4,5-dihydro-3H-2,3-benzodiazepin (V)

In 62 ml of methanol 4.14 g (8.8 mmol) of (S) acetic acid [[2-[2-[(methylsulphonyl)-oxi]-propil)-5-chlorophenyl](3-methyl-4-nitrophenyl)-methylene]hydrazide are dissolved. The solution is cooled between 0-5° C. with an ice-cold water bath and 1.0 ml (10.0 mmol) of 10 n aqueous sodium hydroxide is added. The reaction mixture is stirred for 4 hours, then evaporated in vacuo. The residue was solidified by additional 30 ml of water, then filtered and washed with 5×5 ml of water. The obtained raw product is chromatographed on silica gel using a mixture of hexane and ethylacetate. The product is recrystallized from ethanol. Yield 1.25 g (33.6% is the overall yield calculated isochromane compound). The melting point of the product is 165-167° C.

[α]_{D}=+140.0° (c=1, CHCl₃)

IR (KBr): 1682, 1658, 1503, 1341, 1039 cm⁻¹.

¹H-NMR (CDCl₃, 500 MHz): 8.00 (d, J=8.6 Hz, 1H), 7.53 (m, 2H), 6.76 (s, 1H), 6.49 (s, 1H), 6.02 (s, 2H), 5.36 (m, 1H), 3.00 (dd, J1=3.2 Hz, J2=14.6 Hz, 1H), 2.76 (dd, J1=8.5 Hz, J2=14.6 Hz, 1H), 2.64 (s, 3H), 2.29 (s, 3H), 1.08 (d, J=6.5 Hz, 3H) ppm.

¹³C-NMR: 171.84, 154.56, 149.32, 146.27, 144.19, 135.40, 133.62, 133.32, 127.66, 125.58, 124.67, 109.54, 109.51, 101.71, 58.20, 38.38, 22.83, 20.59, 18.68 ppm.

### Example 14

### (S)-(−)-3-acetyl-8-chloro-4-methyl-1-(3-methyl-4-nitrophenyl)-4,5-dihydro-3H-2,3-benzodiazepin (V)

### Step A

### (1RS,3R)-7-Chloro-3-methyl-1-(3-methyl-4-nitrophenyl)-izochroman-1-ol (XIII)

A solution of 3.35 g (10.0 mmol) of (1RS,3R)-7-chloro-3-methyl-1-(3-methyl-4-nitrophenyl)-izochromane is dissolved in a mixture of 8 ml of dimethylsulphoxide and 27 ml of dimethylformamide, cooled in an ice-cold water bath, then 1.8 ml (18.0 mmol) of 10 N aqueous sodium hydroxide are added. The reaction mixture is bubbled through by air for 4 hours at room temperature. The reaction mixture is added to a solution of to 67 ml (67 mmol) of 1N hydrochloric acid cooled in an ice-cold water bath. The precipitated product is filtered, washed with water and dried to constant weight (3.59 g).

The product is a mixture of isomers and can be used in the next reaction step without further purification.

IR (KBr): 3333, 1653, 1520, 1347, 1172, 1066 cm⁻¹.

¹H-NMR (CDCl₃, 500 MHz): 7.96 (d, J=9.2 Hz, 1H), 7.56 (m, 2H), 7.18 (dd, J1=2.2 Hz, J2=8.3 Hz, 1H), 7.09 (d, J=8.2 Hz, 1H), 6.96 (d, J=2.2 Hz, 1H), 4.45 (m, 1H), 2.80 (m, 2H), 2.61 (s, 3H), 1.43 (d, J=6.2 Hz, 3H) ppm.

¹³C-NMR (CDCl₃): 149.38, 138.49, 133.72, 132.33, 132.18, 130.55, 129.97, 128.98, 128.47, 127.88, 124.84, 124.79, 97.26, 65.70, 35.55, 21.30, 20.70 ppm.

### Step B

### (R) acetic acid [[5-chloro-2-(2-hydroxypropyl)-phenyl]-(3-methyl-4-nitrophenyl)-methylene]hydrazide (XIV)

To a solution of 3.52 g (10.0 mmol) of (1RS,3R)-7-Chloro-3-methyl-1-(3-methyl-4-nitrophenyl)-izochroman-1-ol prepared in step A in 25 ml of isopropanol, 1.16 g (15.7 mmol) of acetic acid hydrazide and 0.23 ml (2.8 mmol) of concentrated hydrochloric acid are added.

The solution is boiled for 6 hours, then cooled to room temperature and the solvent is evaporated in vacuo. The residue is dissolved in 100 ml of ethylacetate, then the obtained solution is washed with 50 ml of saturated sodium hydrogen carbonate solution, then 3×50 of saturated sodium chloride solution and dried with anhydrous sodium sulphate. Following the filtration of the drying agent, the solvent is evaporated in vacuo. The product is a yellow oil (3.69 g, 95%), which is a mixture of E and Z isomers in the ratio of about 1:1. The product may be used in the next reaction step without further purification.

IR (KBr): 3423, 1670, 1518, 1339, 732 cm⁻¹.

¹H-NMR (CDCl₃, 500 MHz): 7.95 (m, 1H), 7.46 (m, 4H), 7.15 (m, 0.5H), 7.09 (m, 0.5H), 3.93 (m, 0.5H), 3.71 (m, 0.5H), 5.59 (s, 3H), 2.42 (s, 1.5H), 2.39 (m, 2H), 2.35 (s, 1.5H), 1.14 (d, J=6.1 Hz, 1.5H), 1.13 (d, J=6.2 Hz, 1.5H) ppm.

### Step C

### (R) acetic acid [[2-[2-[(methylsulphonyl)-oxi]-propyl)-5-chlorophenyl](3-methyl-4-nitrophenyl)-methylene]hydrazide (XIV)

To a solution of 3.69 g (9.46 mmol) of (R) acetic acid [[5-chloro-2-(2-hydroxypropyl)-phenyl]-(3-methyl-4-nitrophenyl)-methylene]hydrazide prepared according to step B in 30 ml of dichloromethane, 1.93 ml (15 mmol) of triethylamine are added, then the reaction mixture is cooled between 0-5° C. To the reaction mixture 0.86 ml (11.0 mmol) of methanesulphonyl chloride is added dropwise and the reaction mixture is kept cold and stirred for additional 4 hours. Then the mixture is diluted with 30 ml of dichloromethane and washed with 25 ml of water, 25 ml of 1 N hydrochloric acid, 3×25 ml of saturated sodium chloride solution, dried over sodium sulphate, then the solvent is evaporated in vacuo. Thus, the product is 4.12 g (93%) of yellow oil as a mixture of optical active hydrazides, which are used in the next reaction step without further purification.

IR (KBr): 3181, 1683, 1520, 1330, 1172 cm⁻¹.

¹H-NMR (CDCl₃, 500 MHz): 8.33 (bs, 0.4*1H), 8.30 (bs, 0.6*1H), 7.38-7.56 (m, 4H), 7.16 (d, J=2.2 Hz, 0.4*1H), 7.14 (d, J=2.2 Hz, 0.6*1H), 4.83 (m, 1H), 2.90 (s, 0.6*3H), 2.83 (s, 0.4*1H), 2.65 (m, 2H), 2.60 (s, 0.4*3H), 2.59 (s, 0.6*3H), 2.48 (s, 0.6*3H), 2.47 (s, 0.4*3H), 1.32 (d, J=6.5 Hz, 0.4*3H), 1.31 (d, J=6.3 Hz, 0.4*3H) ppm.

### Step D

### (S)-(−)-3-acetil-8-chloro-4-methyl-1-(3-methyl-4-nitrophenyl)-4,5-dihydro-3H-2,3-benzodiazepin (V)

In 62 ml of methanol 4.12 g (8.8 mmoles) of (R) acetic acid [[2-[2-[(methylsulphonyl)-oxi]-propil)-5-chlorophenyl](3-methyl-4-nitrophenyl)-methylene]hydrazide are dissolved. The solution is cooled between 0-5° C. in an ice-cold water bath and 1.0 ml (10.0 mmoles) of 10 N aqueous sodium hydroxide is added. The reaction mixture is stirred for 4 hours, then evaporated in vacuo. The residue was solidified by additional 30 ml of water, then filtered and washed with 5×5 ml of water. The obtained raw product is chromatographed on silica gel using a mixture of hexane and ethylacetate. The product is recrystallized from ethanol. Thus, the product weighs 1.28 g (34.4% overall yield based on isochromane compound). The melting point of the product is 164-167° C.

[α]_{D}=−138.6° (c=1, CHCl₃)

IR (KBr): 1682, 1658, 1503, 1341, 1039 cm⁻¹.

¹H-NMR (CDCl₃, 500 MHz): 8.00 (d, J=8.6 Hz, 1H), 7.53 (m, 2H), 6.76 (s, 1H), 6.49 (s, 1H), 6.02 (s, 2H), 5.36 (m, 1H), 3.00 (dd, J1=3.2 Hz, J2=14.6 Hz, 1H), 2.76 (dd, J1=8.5 Hz, J2=14.6 Hz, 1H), 2.64 (s, 3H), 2.29 (s, 3H), 1.08 (d, J=6.5 Hz, 3H) ppm.

¹³C-NMR: 171.84, 154.56, 149.32, 146.27, 144.19, 135.40, 133.62, 133.32, 127.66, 125.58, 124.67, 109.54, 109.51, 101.71, 58.20, 38.38, 22.83, 20.59, 18.68 ppm.

### Example 15

### (R)-(−)-3-acetyl-1-(4-amino-3-methylphenyl)-8-chloro-4-methyl-4,5-dihydro-3H-2,3-benzodiazepine (I/R)

In a mixture of 20 ml of methanol and 6 ml of dichloromethane 1.15 g (3.1 mmoles) of (R)-(+)-3-acetyl-8-chloro-4-methyl-1-(3-methyl-4-nitrophenyl)-4,5-dihydro-3H-2,3-benzodiazepine (according to Example 3) is dissolved. About 0.5 g of wet Raney-nickel catalyst, then 0.54 ml (11.1 mmol) of 98% hydrazine hydrate are added to the reaction mixture under vigorous stirring.

The reduction takes place during one hour accompanied by intensive gas evolution and slight elevation of the temperature of the reaction mixture.

Following the completion of the reduction, the catalyst is filtered off from the reaction mixture, the reaction mixture is evaporated in vacuo and the raw product is triturated with 20 ml of water and solidified.

Thus, the product weighs 0.92 g (87%). The melting point is 100-103° C. The optical purity of the product is higher than 99.7 e.e. (determined by chiral HPLC).

[α]_{D}²⁰=−637.3° (c=1, CHCl₃)

IR (KBr): 3453, 3335, 3222, 1625 cm⁻¹.

¹H-NMR (CDCl₃, 400 MHz): 7.48 (d, J=1.3 Hz, 1H), 7.35 (dd, J1=2.1 Hz, J2=8.1 Hz, 1H), 7.28 (dd, J1=2.0 Hz, J2=8.2 Hz, 1H), 7.22 (d, J=78.2 Hz, 1H), 7.12 (d, J=2.2 Hz, 1H), 6.67 (d, J=8.3 Hz, 1H), 5.21 (m, 1H), 4.01 (bs, 2H), 2.79 (dd, J1=5.5 Hz, J2=13.7 Hz, 1H), 2.65 (dd, J1=12.0 Hz, J2=13.6 Hz, 1H), 2.20 (s, 3H), 1.30 (d, J=6.4 Hz, 3H) ppm.

¹³C-NMR: 172.14, 169.21, 148.14, 138.46, 135.83, 132.35, 131.43, 130.27, 129.40, 129.24, 128.72, 125.45, 121.79, 114.03, 60.47, 38.28, 22.60, 18.32, 17.32 ppm.

### Example 16

### (S)-(+)-3-acetyl-1-(4-amino-3-methylphenyl)-8-chloro-4-methyl-4,5-dihydro-3H-2,3-benzodiazepine (I/S)

In a mixture of 20 ml of methanol and 6 ml of dichloromethane 1.15 g (3.1 mmoles) of (S)-(−)-3-acetyl-8-chloro-4-methyl-1-(3-methyl-4-nitrophenyl)-4,5-dihydro-3H-2,3-benzodiazepine (according to Example 4) is dissolved. About 0.5 g of wet Raney-nickel catalyst, then 0.54 ml (11.1 mmoles) of 98% hydrazine hydrate are added to the reaction mixture under vigorous stirring.

The reduction takes place in one hour, accompanied by intense gas evolution and slight elevation of temperature of the reaction mixture.

Following the completion of the reduction, the catalyst is filtered off from the reaction mixture, the reaction mixture is evaporated in vacuo and the raw product is triturated with 20 ml of water and solidified.

Thus, the yield is 0.94 g (89%). The melting point is 100-103° C. The optical purity of the product is higher than 99.7 e.e. (determined by chiral HPLC).

[α]_{D}²⁰=+635.1° (c=1, CHCl₃)

IR (KBr): 3453, 3335, 3222, 1625 cm⁻¹.

¹H-NMR (CDCl₃, 400 MHz): 7.48 (d, J=1.3 Hz, 1H), 7.35 (dd, J1=2.1 Hz, J2=8.1 Hz, 1H), 7.28 (dd, J1=2.0 Hz, J2=8.2 Hz, 1H), 7.22 (d, J=78.2 Hz, 1H), 7.12 (d, J=2.2 Hz, 1H), 6.67 (d, J=8.3 Hz, 1H), 5.21 (m, 1H), 4.01 (bs, 2H), 2.79 (dd, J1=5.5 Hz, J2=13.7 Hz, 1H), 2.65 (dd, J1=12.0 Hz, J2=13.6 Hz, 1H), 2.20 (s, 3H), 1.30 (d, J=6.4 Hz, 3H) ppm.

¹³C-NMR: 172.14, 169.21, 148.14, 138.46, 135.83, 132.35, 131.43, 130.27, 129.40, 129.24, 128.72, 125.45, 121.79, 114.03, 60.47, 38.28, 22.60, 18.32, 17.32 ppm.

### B. Processes Via dihydro-2,3-benzodiazepine Derivatives Acylated with Dicarboxylic Acids

### Example 17

### 4-(8-Methyl-5-(3-methyl-4-nitrophenyl)-8,9-dihydro-7H-1,3-dioxolo[4,5-h][2,3]benzodiazepine-7-yl)-4-oxo-but-2-en carboxylic acid (III)

A solution containing 6.2 g (0.063 mmoles) of maleic anhydride in 50 ml of dichloromethane is added drop by drop in 30 minutes to a mixture of 9.8 g (0.0289 moles) of 8-methyl-1-(3-methyl-4-nitrophenyl)-8,9-dihydro-7H-1,3-dioxolo[4,5-h][2,3]benzodiazepine, 5 ml (0.0357 moles) of triethylamine and 80 ml of dichloromethane between 3-6° C. temperature. Then the reaction mixture is stirred at reflux temperature for 3 hours and cooled to room temperature. The organic layer is extracted with 3×25 ml of 5% aqueous hydrochloric acid and 2×30 ml of water, then dried over magnesium sulphate and evaporated in vacuo. Hexane is added to the residue and evaporated in vacuum, then diisopropylether is added and evaporated again. To the residue 80 ml of diisopropylether is added and stirred for two hours. The obtained crystals are filtered and washed with diisopropylether and dried under infra lamp.

Thus, the yield is 10.88 g (86.2%) of the title product.

The melting point is 158-160° C.

IR (KBr): 3450, 3091, 2415, 1720, 1341 cm⁻¹.

¹H-NMR (CDCl₃, i400): 8.02 (d, J=8.2 Hz, 1H), 7.61 (m, 2H), 6.97 (d, J=13.1 Hz, 1H), 6.85 (s, 1H), 6.49 (s, 1H), 6.37 (d, J=13.1 Hz, 1H), 6.08 (d, J=1.3 Hz, 1H), 6.05 (d, J=1.3 Hz, 1H), 5.37 (m, 1H), 2.90 (dd, J1=4.7 Hz, J2=14.4 Hz, 1H), 2.75 (dd, J1=11.3 Hz, J2=14.3 Hz, 1H), 2.65 (s, 3H), 1.35 (d, J=6.4 Hz, 3H) ppm.

¹³C-NMR (CDCl₃, i400): 169.52, 165.05, 163.72, 150.78, 150.52, 146.99, 140.40, 135.34, 134.78, 133.90, 133.67, 128.31, 128.09, 124.93, 124.85, 109.25, 109.14, 102.13, 62.87, 37.97, 20.39, 18.28 ppm.

### Example 18

### Salt of (S)-(+)-4-(8-Methyl-5-(3-methyl-4-nitrophenyl)-8,9-dihydro-7H-1,3-dioxolo[4,5-h] [2,3] benzodiazepine-7-yl)-4-oxo-but-2-en carboxylic acid with R-(+)-α-methyl-benzylamine (IV)

To a solution of 5.4 g (0.0123 moles) of 4-(8-Methyl-5-(3-methyl-4-nitrophenyl)-8,9-dihydro-7H-1,3-dioxolo[4,5-h] [2,3] benzodiazepine-7-yl)-4-oxo-but-2-en carboxylic acid (III) in 310 ml of ethylacetate 1.57 ml of R-(+)-α-methyl-benzylamine are added at room temperature. Following a two-hour stirring at room temperature, the precipitated crystals are filtered, washed with ethylacetate and diethylether. The obtained raw product is recrystallized from ethylacetate.

Thus, the yield is 2.48 g (72%) of the title product.

Melting point 148-150° C.

[α]²⁰_{D}: +122.6° (c=1, CH₃OH)

IR (KBr): 3440, 2974, 1670, 1625, 1517, 1342, 1039 cm⁻¹.

¹H-NMR (CDCl₃, i400): 8.01 (d, J=8.3 Hz, 1H), 7.60 (m, 2H), 7.44 (m, 1H), 7.32 (m, 4H), 6.86 (d, J=12.9 Hz, 1H), 6.82 (s, 1H), 6.48 (s, 1H), 6.31 (d, J=12.9 Hz, 1H), 6.06 (d, J=1.2 Hz, 1H), 6.04 (d, J=1.2 Hz, 1H), 5.31 (m, 1H), 4.32 (q, J=6.8 Hz, 1H), 2.89 (dd, J1=4.4 Hz, J2=14.5 Hz, 1H), 2.73 (dd, J1=10.9 Hz, J2=14.5 Hz, 1H), 2.64 (s, 3H), 1.62 (d, J=6.9 Hz, 1H), 1.28 (d, J=6.5 Hz, 3H) ppm.

### Example 19

### Salt of (R)-(−)-4-(8-Methyl-5-(3-methyl-4-nitrophenyl)-8,9-dihydro-7H-1,3-dioxolo[4,5-h] [2,3] benzodiazepine-7-yl)-4-oxo-but-2-en carboxylic acid with S-(−)-α-methyl-benzylamine (IV)

To a solution of 5.4 g (0.0123 moles) of 4-(8-Methyl-5-(3-methyl-4-nitrophenyl)-8,9-dihydro-7H-1,3-dioxolo[4,5-h] [2,3] benzodiazepine-7-yl)-4-oxo-but-2-en carboxylic acid (III) in 310 ml of ethylacetate 1.57 ml of R-(+)-α-methyl-benzylamine are added at room temperature. Following a two-hour stirring at room temperature, the precipitated crystals are filtered, washed with ethylacetate and diethylether. The obtained raw product is recrystallized from ethylacetate.

Thus, the yield is 2.45 g (70.6%) of the title product.

Melting point: 148-150° C.

[α]²⁰_{D}: −124.1° (c=1, CH₃OH)

IR (KBr): 3440, 2974, 1670, 1625, 1517, 1342, 1039 cm⁻¹.

¹H-NMR (CDCl₃, i400): 8.01 (d, J=8.3 Hz, 1H), 7.60 (m, 2H), 7.44 (m, 1H), 7.32 (m, 4H), 6.86 (d, J=12.9 Hz, 1H), 6.82 (s, 1H), 6.48 (s, 1H), 6.31 (d, J=12.9 Hz, 1H), 6.06 (d, J=1.2 Hz, 1H), 6.04 (d, J=1.2 Hz, 1H), 5.31 (m, 1H), 4.32 (q, J=6.8 Hz, 1H), 2.89 (dd, J1=4.4 Hz, J2=14.5 Hz, 1H), 2.73 (dd, J1=10.9 Hz, J2=14.5 Hz, 1H), 2.64 (s, 3H), 1.62 (d, J=6.9 Hz, 1H), 1.28 (d, J=6.5 Hz, 3H) ppm.

### Example 20

### (S)-(+)-4-(8-Methyl-5-(3-methyl-4-nitrophenyl)-8,9-dihydro-7H-1,3-dioxolo[4,5-h] [2,3] benzodiazepine-7-yl)-4-oxo-but-2-ene carboxylic acid (III/A)

9.3 g (0.0167 moles) of (S)-(+)-4-(8-Methyl-5-(3-methyl-4-nitrophenyl)-8,9-dihydro-7H-1,3-dioxolo[4,5-h] [2,3] benzo-diazepine-7-yl)-4-oxo-but-2-ene carboxylic acid R-(+)-α-methyl-benzylamine salt is stirred in 94 ml of dichloro-methane and to this mixture 94 ml of a mixture of concentrated hydrochloric acid and water in the ratio of 1:1 are added. The mixture is left stirred for further 15 minutes. The organic layer is separated, washed with 2×70 ml of a mixture of concentrated hydrochloric acid and water in the ratio of 1:1, then with 2×150 ml of water. The organic phase is dried over magnesium sulphate, evaporated in vacuo, 2×100 ml of hexane are added to the residue and evaporated in vacuo. The residue is stirred with 125 ml of diisopropylether at room temperature, and the precipitated crystals are filtered and washed with 3×30 ml of diisopropylether and dried under infrared lamp.

Thus, the yield is 6.13 g (84.2%) of the title product.

Melting point: 170-172° C.

[α]²⁰_{D}: +349.5° (c=1, CHCl₃)

IR (KBr): 3450, 3091, 2415, 1720, 1341 cm⁻¹.

¹H-NMR (CDCl₃, i400): 8.02 (d, J=8.2 Hz, 1H), 7.61 (m, 2H), 6.97 (d, J=13.1 Hz, 1H), 6.85 (s, 1H), 6.49 (s, 1H), 6.37 (d, J=13.1 Hz, 1H), 6.08 (d, J=1.3 Hz, 1H), 6.05 (d, J=1.3 Hz, 1H), 5.37 (m, 1H), 2.90 (dd, J1=4.7 Hz, J2=14.4 Hz, 1H), 2.75 (dd, J1=11.3 Hz, J2=14.3 Hz, 1H), 2.65 (s, 3H), 1.35 (d, J=6.4 Hz, 3H) ppm.

¹³C-NMR (CDCl₃, i400): 169.52, 165.05, 163.72, 150.78, 150.52, 146.99, 140.40, 135.34, 134.78, 133.90, 133.67, 128.31, 128.09, 124.93, 124.85, 109.25, 109.14, 102.13, 62.87, 37.97, 20.39, 18.28 ppm.

### Example 21

### (R)-(−)-4-(8-Methyl-5-(3-methyl-4-nitrophenyl)-8,9-dihydro-7H-1,3-dioxolo[4,5-h] [2,3] benzodiazepine-7-yl)-4-oxo-but-2-ene carboxylic acid (III/A)

9.3 g (0.0167 moles) of (R)-(−)-4-(8-Methyl-5-(3-methyl-4-nitrophenyl)-8,9-dihydro-7H-1,3-dioxolo[4,5-h] [2,3] benzodiazepine-7-yl)-4-oxo-but-2-ene carboxylic acid S-(−)-α-methyl-benzylamine salt is stirred in 94 ml of dichloro-methane and 94 ml of a mixture of concentrated hydrochloric acid and water in the ratio of 1:1 is added, then the mixture is stirred for further 15 minutes. The organic layer is separated, washed with 2×70 ml of a mixture of concentrated hydrochloric acid and water in the ratio of 1:1, then with 2×150 ml of water. The organic phase is dried over magnesium sulphate, evaporated in vacuum, 2×100 ml of hexane are added to the residue and evaporated in vacuum. The residue is stirred with 125 ml of diisopropylether at room temperature, and the precipitated crystals are filtered and washed with 3×30 ml of diisopropylether and dried under infrared lamp.

Thus, the yield is 6.20 g (85.2%) of the title product.

Melting point: 170-172° C.

[α]²⁰_{D}: −350.2° (c=1, CHCl₃)

IR (KBr): 3450, 3091, 2415, 1720, 1341 cm⁻¹.

¹H-NMR (CDCl₃, i400): 8.02 (d, J=8.2 Hz, 1H), 7.61 (m, 2H), 6.97 (d, J=13.1 Hz, 1H), 6.85 (s, 1H), 6.49 (s, 1H), 6.37 (d, J=13.1 Hz, 1H), 6.08 (d, J=1.3 Hz, 1H), 6.05 (d, J=1.3 Hz, 1H), 5.37 (m, 1H), 2.90 (dd, J1=4.7 Hz, J2=14.4 Hz, 1H), 2.75 (dd, J1=11.3 Hz, J2=14.3 Hz, 1H), 2.65 (s, 3H), 1.35 (d, J=6.4 Hz, 3H) ppm.

¹³C-NMR (CDCl₃, i400): 169.52, 165.05, 163.72, 150.78, 150.52, 146.99, 140.40, 135.34, 134.78, 133.90, 133.67, 128.31, 128.09, 124.93, 124.85, 109.25, 109.14, 102.13, 62.87, 37.97, 20.39, 18.28 ppm.

### Example 22

### (S)-(−)-8-Methyl-5-(3-methyl-4-nitrophenyl)-8,9-dihydro-7H-1,3-dioxolo[4,5-h] [2,3] benzodiazepine (II/A)

To a solution of 6.0 g (0.0137 moles) of (S)-(+)-4-(8-Methyl-5-(3-methyl-4-nitrophenyl)-8,9-dihydro-7H-1,3-dioxolo[4,5-h] [2,3] benzodiazepine-7-yl)-4-oxo-but-2-en carboxylic acid in 136 ml of tetrahydrofurane, 36 ml of water and 15.2 ml of 30% hydrogen peroxide solution are added. Then a solution of 4.8 g of lithium hydroxyde dissolved in 51 ml of water are added to the mixture and kept at 50° C. for two hours. Having finished the reaction, the organic phase is evaporated in vacuo, then the aqueous phase is washed three times with dichloromethane. The combined organic phases are washed with an 5% aqueous solution of sodium carbonate and with water, dried over magnesium sulphate. The phase containing dichloromethane is evaporated in vacuo, the residue is boiled for half an hour in methanol, cooled with ice-water and the crystals are filtered.

Thus, the yield is 3.21 g (69.2%) of the title product.

Melting point: 152-155° C.

[α]²⁰_{D}=−152.9° (c=1, CHCl₃)

IR (KBr): 1035, 1250, 1335, 1504, 3386 cm⁻¹.

¹H-NMR (CDCl₃): 7.96 (d, J=8.5 Hz, 1H), 7.53 (m, 1H), 7.45 (m, 1H), 6.74 (s, 1H), 6.50 (s, 1H), 5.98 (s, 2H), 5.59 (bs, 1H), 4.09 (m, 1H), 2.86 (dd, J1=4.0 Hz, J2=13.9 Hz, 1H), 2.64 (dd, J1=6.4 Hz, J2=14.0 Hz, 1H), 2.61 (s, 3H), 1.27 (d, J=6.4 Hz, 3H) ppm.

¹³C-NMR: 150.25, 148.28, 145.86, 144.74, 135.57, 133.69, 132.40, 126.83, 126.78, 124.67, 109.05, 108.87, 101.35, 63.93, 40.10, 22.00, 20.72 ppm.

### Example 23

### (R)-(+)-8-Methyl-5-(3-methyl-4-nitrophenyl)-8,9-dihydro-7H-1,3-dioxolo[4,5-h] [2,3] benzodiazepine (II/A)

To a solution of 6.0 g (0.0137 moles) of (R)-(−)-4-(8-Methyl-5-(3-methyl-4-nitrophenyl)-8,9-dihydro-7H-1,3-dioxolo[4,5-h] [2,3] benzodiazepine-7-yl)-4-oxo-but-2-en carboxylic acid in 136 ml of tetrahydrofurane, 36 ml of water and 15.2 ml of 30% hydrogen peroxide solution are added. Then 4.8 g of lithium hydroxide dissolved in 51 ml of water are added to the mixture and it is kept at 50° C. for two hours. Having finished the reaction, the organic phase is evaporated in vacuo, then the aqueous phase is washed three times with dichloromethane. The combined organic phases are washed with 5% aqueous solution of sodium carbonate and with water and dried over magnesium sulphate. The phase containing dichloromethane is evaporated in vacuo, the residue is boiled for half an hour in methanol, cooled with ice-water and the crystals are filtered.

Thus, the yield is 3.16 g (68.1%) of the titled product.

Melting point: 152-155° C.

[α]²⁰_{D}=+152.9° (c=1, CHCl₃)

IR (KBr): 1035, 1250, 1335, 1504, 3386 cm⁻¹

¹H-NMR (CDCl₃): 7.96 (d, J=8.5 Hz, 1H), 7.53 (m, 1H), 7.45 (m, 1H), 6.74 (s, 1H), 6.50 (s, 1H), 5.98 (s, 2H), 5.59 (bs, 1H), 4.09 (m, 1H), 2.86 (dd, J1=4.0 Hz, J2=13.9 Hz, 1H), 2.64 (dd, J1=6.4 Hz, J2=14.0 Hz, 1H), 2.61 (s, 3H), 1.27 (d, J=6.4 Hz, 3H) ppm.

¹³C-NMR: 150.25, 148.28, 145.86, 144.74, 135.57, 133.69, 132.40, 126.83, 126.78, 124.67, 109.05, 108.87, 101.35, 63.93, 40.10, 22.00, 20.72 ppm.

### Example 24

### (S)-(+)-7-Acetyl-8-methyl-5-(3-methyl-4-nitrophenyl)-8,9-dihydro-7H-1,3-dioxolo[4,5-h] [2,3] benzodiazepine (V)

1.70 g (5.0 mmoles) of (S)-(−)-8-Methyl-5-(3-methyl-4-nitrophenyl)-8,9-dihydro-7H-1,3-dioxolo[4,5-h][2,3]benzodiazepine and 10 ml of acetic anhydride are stirred at room temperature for 24 hours. After stirring the mixture is poured into a mixture of 100 ml of water and 75 ml of dichloromethane. The resulting mixture is stirred for an hour, then sodium carbonate is added in small portions until the pH value is raised to 8. The layers are separated and the aqueous layer is extracted twice with 25 ml of dichloromethane. The combined organic layers are washed with 50 ml of saturated sodium chloride solution, dried over magnesium sulphate, then evaporated. The obtained raw product is recrystallized from 15 ml of methanol.

Thus the yield of the desired recrystallised product is 1.62 g (85%).

Melting point: 124-128° C.

[α]²⁰_{D}=+44.2° (c=1, CHCl₃)

IR (KBr): 1682, 1658, 1503, 1341, 1039 cm⁻¹.

¹H-NMR (CDCl₃): 8.00 (d, J=8.6 Hz, 1H), 7.53 (m, 2H), 6.76 (s, 1H), 6.49 (s, 1H), 6.02 (s, 2H), 5.36 (m, 1H), 3.00 (dd, J1=3.2 Hz, J2=14.6 Hz, 1H), 2.76 (dd, J1=8.5 Hz, J2=14.6 Hz, 1H), 2.64 (s, 3H), 2.29 (s, 3H), 1.08 (d, J=6.5 Hz, 3H) ppm

¹³C-NMR: 171.84, 154.56, 149.32, 146.27, 144.19, 135.40, 133.62, 133.32, 127.66, 125.58, 124.67, 109.54, 109.51, 101.71, 58.20, 38.38, 22.83, 20.59, 18.68 ppm.

### Example 25

### (R)-(−)-7-Acetyl-8-methyl-5-(3-methyl-4-nitrophenyl)-8,9-dihydro-7H-1,3-dioxolo[4,5-h] [2,3] benzodiazepine (V)

A mixture of 1.70 g (5.0 mmoles) of (R)-(+)-8-Methyl-5-(3-methyl-4-nitrophenyl)-8,9-dihydro-7H-1,3-dioxolo[4,5-h][2,3]-benzodiazepine and 10 ml of acetic anhydride are stirred at room temperature for 24 hours. Then the mixture is poured into a mixture of 100 ml of water and 75 ml of dichloromethane and the resulting mixture is stirred for an hour. Thereafter sodium carbonate is added in small portions to the mixture until the pH value is raised to 8. The layers are separated and the aqueous layer is extracted twice with 25 ml of dichloromethane. The combined organic layers are washed with 50 ml of saturated sodium chloride solution, dried over magnesium sulphate, then evaporated. The obtained raw product is recrystallized from 15 ml of methanol.

Thus, the recrystallised title product is 1.61 g (85%).

Melting point: 124-128° C.

[α]²⁰_{D}=−44.4° (c=1, CHCl₃)

IR (KBr): 1682, 1658, 1503, 1341, 1039 cm⁻¹.

¹H-NMR (CDCl₃): 8.00 (d, J=8.6 Hz, 1H), 7.53 (m, 2H), 6.76 (s, 1H), 6.49 (s, 1H), 6.02 (s, 2H), 5.36 (m, 1H), 3.00 (dd, J1=3.2 Hz, J2=14.6 Hz, 1H), 2.76 (dd, J1=8.5 Hz, J2=14.6 Hz, 1H), 2.64 (s, 3H), 2.29 (s, 3H), 1.08 (d, J=6.5 Hz, 3H) ppm.

¹³C-NMR: 171.84, 154.56, 149.32, 146.27, 144.19, 135.40, 133.62, 133.32, 127.66, 125.58, 124.67, 109.54, 109.51, 101.71, 58.20, 38.38, 22.83, 20.59, 18.68. ppm

### Example 26

### (S)-(+)-8-methyl-5-(3-methyl-4-nitrophenyl)-7-propionyl-8,9-dihydro-7H-1,3-dioxolo[4,5-h][2,3] benzodiazepine (V)

1.70 g (5.0 mmoles) of (S)-(−)-8-Methyl-5-(3-methyl-4-nitrophenyl)-8,9-dihydro-7H-1,3-dioxolo[4,5-h][2,3]benzodiazepine and 10 ml of propionic acid anhydride are stirred at room temperature for 24 hours. Then the mixture is poured into a mixture of 100 ml of water and 75 ml of dichloromethane and the resulting mixture is stirred for an hour, then sodium carbonate is added in small portions to the mixture until the pH value is raised to 8. The layers are separated and the aqueous layer is extracted twice with 25 ml of dichloromethane. The combined organic layers are washed with 50 ml of saturated sodium chloride solution, dried over magnesium sulphate then evaporated. The obtained raw product is used without further purification.

¹H-NMR (CDCl₃): 8.00 (1H, d, J=9.6 Hz), 7.54 (2H, m), 6.77 (1H, s), 6.49 (1H, s), 6.01 (2H, s), 5.37 (1H, m), 2.98 (1H, dd, J=14.5 és J=3.4 Hz), 2.76 (1H, dd, J=14.6 és J=8.7 Hz), 2.66 (2H, m), 2.64 (3H, s), 1.14 (3H, t, J=7.4 Hz), 1.09 (3H, d, J=6.5 Hz) ppm.

### Example 27

### (R)-(−)-8-methyl-5-(3-methyl-4-nitrophenyl)-7-propionyl-8,9-dihydro-7H-1,3-dioxolo[4,5-h][2,3] benzodiazepine (V)

1.70 g (5.0 mmoles) of (R)-(+)-8-Methyl-5-(3-methyl-4-nitrophenyl)-8,9-dihydro-7H-1,3-dioxolo[4,5-h][2,3]benzo-diazepine and 10 ml of propionic acid anhydride are stirred at room temperature for 24 hours. Then the mixture is poured into a mixture of 100 ml of water and 75 ml of dichloromethane and the resulting mixture is stirred for an hour, then sodium carbonate is added in small portions to the mixture until the pH value is raised to 8. The layers are separated and the aqueous layer is extracted twice with 25 ml of dichloromethane. The combined organic layers are washed with 50 ml of saturated sodium chloride solution, dried over magnesium sulphate then evaporated. The obtained raw product may be used without further purification.

¹H-NMR (CDCl₃): 8.00 (1H, d, J=9.6 Hz), 7.54 (2H, m), 6.77 (1H, s), 6.49 (1H, s), 6.01 (2H, s), 5.37 (1H, m), 2.98 (1H, dd, J=14.5 és J=3.4 Hz), 2.76 (1H, dd, J=14.6 és J=8.7 Hz), 2.66 (2H, m), 2.64 (3H, s), 1.14 (3H, t, J=7.4 Hz), 1.09 (3H, d, J=6.5 Hz) ppm.

Reduction of the nitro compounds according to the Examples 19, 18, 20 and 21 can be carried out as it is described in the corresponding Examples (4, 6, 8 and 10).

### C. Separation Via Diastereomer Salt Composed with Optically Active Amines

### Example 28

### (±)-8-Methyl-5-(3-methyl-4-nitrophenyl)-8,9-dihydro-7H-1,3-dioxolo[4,5-h][2,3] benzodiazepine-7-carboxylic acid-imidazolide (VI)

A mixture of 3.37 g (10.0 mmoles) of (±)-8-Methyl-5-(3-methyl-4-nitrophenyl)-8,9-dihydro-7H-1,3-dioxolo[4,5-h] [2,3]benzodiazepine, 1.95 g (12.0 mmoles) of 1,1′-carbonyl-diimidazole and 20 ml of anhydrous tetrahydrofurane are refluxed for 15 hours. Then the reaction mixture is cooled with ice-water, the precipitated product is filtered and washed with 10 ml of diethylether.

Thus, the yield is 3.90 g (90%) of the title product.

Melting point: 223-226° C.

¹H-NMR ((CD₃)₂SO): 8.06 (d, J=8.5 Hz, 1H), 7.96 (s, 1H), 7.57 (s, 1H), 7.54 (dd, J=8.5 Hz és J=1.5 Hz, 1H), 7.38 (s, 1H), 7.04 (s, 1H), 7.13 (s, 1H), 6.87 (s, 1H), 6.13 (d, J=0.8 Hz, 1H), 6.10 (d, J=0.9 Hz, 1H), 5.08 (m, 1H), 3.30 (s, 1H), 3.05 (dd, J=14.3 és J=5.0 Hz, 1H), 2.73 (dd, J=14.2 és 10.2 Hz, 1H), 1.30 (d, J=6.2 Hz, 3H) ppm.

### Example 29

### (+)-7-(N-(1(R)-phenylethyl)-carbamoyl)-8(R)-methyl-5-(3-methyl-4-nitrophenyl)-8,9-dihydro-7H-1,3-dioxolo[4,5-h][2,3]benzodiazepine (VII)

To a suspension of 11.08 g (25.0 mmoles) (±)-8-Methyl-5-(3-methyl-4-nitrophenyl)-8,9-dihydro-7H-1,3-dioxolo[4,5-h][2,3]benzodiazepine-7-carboxylic acid-imidazolide in 75 ml of anhydrous dimethylformamide 6.64 g, 3.82 ml (30.0 mmoles) of (R)-phenylethylamine are added. The reaction mixture is stirred for 24 hours using an oil bath at 110° C. Then the solvent is evaporated at 55 Pa pressure, the residue is dissolved in 200 ml of dichloromethane. The organic phase is washed with 75 ml of water, 75 ml of 6N HCl solution, 75 ml of water, 75 ml of saturated aqueous sodium chloride solution, then dried over magnesium sulphate and evaporated in vacuo. The thus obtained product is boiled for three hours in 150 ml of ethanol, allowed to cool and filtered. The filtrated product of 5.22 g (84%), having a melting point of 201-204° C., is refluxed for three hours in 100 ml of ethanol, then cooled and filtered.

Thus, the yield is 5.00 g (82%) of the title product.

Melting point: 202-205° C.,

[α]²⁰_{D}=+157.3° (c=1, CHCl₃)

IR (KBr): 3420, 1688, 1482, 1353, 1037 cm⁻¹.

¹H-NMR (CDCl₃): 7.97 (d, J=8.4 Hz, 1H), 7.36 (m, 7H), 6.99 (bd, J=8.0 Hz, 1H), 6.71 (s, 1H), 6.48 (s, 1H), 6.00 (d, J=1.3 Hz, 1H), 5.98 (d, J=1.3 Hz, 1H), 5.42 (m, 1H), 5.02 (˜qn, J=7.3 Hz, 1H), 3.13 (dd, J1=1.6 Hz, J2=14.7 Hz, 1H), 2.87 (dd, J1=6.4 Hz, J2=14.7 Hz, 1H), 2.58 (s, 3H), 1.50 (d, J=6.9 Hz, 3H), 0.93 (d, J=6.6 Hz, 3H) ppm.

¹³C-NMR: 155.08, 148.90, 148.44, 146.60, 146.03, 144.18, 142.23, 135.43, 133.80, 133.12, 128.71, 127.33, 127.24, 125.83, 125.46, 124.71, 110.15, 109.99, 101.62, 55.46, 50.15, 39.04, 22.98, 20.65, 19.64 ppm.

### Example 30

### (−)-7-(N-(1(S)-phenylethyl-carbamoyl)-8(S)-methyl-5-(3-methyl-4-nitrophenyl)-8,9-dihydro-7H-1,3-dioxolo[4,5-h][2,3]benzodiazepine (VII)

Following the molar ratios, reaction conditions and process for the work-up of the reaction mixture described in Example 29, and using (S)-phenylethylamine as a chiral base, 5.10 g (82%) of the title product is obtained. Melting point: 202-204° C.

[α]²⁰_{D}=−157.7° (c=1, CHCl₃)

IR (KBr): 3420, 1688, 1482, 1353, 1037 cm⁻¹.

¹H-NMR (CDCl₃): 7.97 (d, J=8.4 Hz, 1H), 7.36 (m, 7H), 6.99 (bd, J=8.0 Hz, 1H), 6.71 (s, 1H), 6.48 (s, 1H), 6.00 (d, J=1.3 Hz, 1H), 5.98 (d, J=1.3 Hz, 1H), 5.42 (m, 1H), 5.02 (˜qn, J=7.3 Hz, 1H), 3.13 (dd, J1=1.6 Hz, J2=14.7 Hz, 1H), 2.87 (dd, J1=6.4 Hz, J2=14.7 Hz, 1H), 2.58 (s, 3H), 1.50 (d, J=6.9 Hz, 3H), 0.93 (d, J=6.6 Hz, 3H) ppm.

¹³C-NMR: 155.08, 148.90, 148.44, 146.60, 146.03, 144.18, 142.23, 135.43, 133.80, 133.12, 128.71, 127.33, 127.24, 125.83, 125.46, 124.71, 110.15, 109.99, 101.62, 55.46, 50.15, 39.04, 22.98, 20.65, 19.64 ppm.

### Example 31

### (R)-(+)-8-Methyl-5-(3-methyl-4-nitrophenyl)-8,9-dihydro-7H-1,3-dioxolo[4,5-h][2,3] benzodiazepine (II/A)

A mixture of 12.16 g (25.0 mmoles) of (+)-7-(N-(1(R)-phenylethyl)-carbamoyl)-8(R)-methyl-5-(3-methyl-4-nitrophenyl)-8,9-dihydro-7H-1,3-dioxolo[4,5-h][2,3]benzodiazepine and 10 ml of 33% hydrogen bromide in acetic acid solution is stirred at room temperature for 24 hours in a closed vessel. Then the mixture is cooled with ice-water, then 120 ml of saturated aqueous sodium chloride solution and 12 ml of ethylacetate are added. After a 20-minute stirring the precipitated product is filtered. The obtained hydrogen bromide salt is stirred in a mixture of 150 ml of ethylacetate and 150 ml of saturated sodium carbonate solution. The layers are separated, the aqueous layer is washed twice with 75 ml of ethylacetate. The combined organic phases are washed with 50 ml of saturated sodium chloride solution, then dried over magnesium sulphate and the solvent is removed by vacuum distillation. The obtained raw product is boiled for half an hour in 75 ml of methanol and cooled with ice-cool water. The obtained crystals are filtered.

Thus, the yield is 6.36 g (75%) of the title product.

Melting point: 144-147° C.

[α]²⁰_{D}=+163.4° (c=1, CHCl₃)

IR (KBr): 3386, 1504, 1335, 1250, 1035 cm⁻¹.

¹H-NMR (CDCl₃): 7.96 (d, J=8.5 Hz, 1H), 7.53 (m, 1H), 7.45 (m, 1H), 6.74 (s, 1H), 6.50 (s, 1H), 5.98 (s, 2H), 5.59 (bs, 1H), 4.09 (m, 1H), 2.86 (dd, J1=4.0 Hz, J2=13.9 Hz, 1H), 2.64 (dd, J1=6.4 Hz, J2=14.0 Hz, 1H), 2.61 (s, 3H), 1.27 (d, J=6.4 Hz, 3H) ppm.

¹³C-NMR: 150.25, 148.28, 145.86, 144.74, 135.57, 133.69, 132.40, 126.83, 126.78, 124.67, 109.05, 108.87, 101.35, 63.93, 40.10, 22.00, 20.72 ppm.

### Example 32

### (S)-(−)-8-Methyl-5-(3-methyl-4-nitrophenyl)-8,9-dihydro-7H-1,3-dioxolo[4,5-h][2,3] benzodiazepine (II/A)

Following the molar ratios, reaction conditions and the work-up procedure of the reaction mixture described in Example 31, with the exception that as starting substance (−)-7-(N-(1(S)-phenylethyl)-carbamoyl)-8(S)-methyl-5-(3-methyl-4-nitrophenyl)-8,9-dihydro-7H-1,3-dioxolo[4,5-h][2,3]benzo-diazepine is used, the title product can be prepared in a yield of 6.30 g (74%).

Melting point: 143-147° C.

[α]²⁰_{D}=−163.1° (c=1, CHCl₃)

IR (KBr): 3386, 1504, 1335, 1250, 1035 cm⁻¹.

¹H-NMR (CDCl₃): 7.96 (d, J=8.5 Hz, 1H), 7.53 (m, 1H), 7.45 (m, 1H), 6.74 (s, 1H), 6.50 (s, 1H), 5.98 (s, 2H), 5.59 (bs, 1H), 4.09 (m, 1H), 2.86 (dd, J1=4.0 Hz, J2=13.9 Hz, 1H), 2.64 (dd, J1=6.4 Hz, J2=14.0 Hz, 1H), 2.61 (s, 3H), 1.27 (d, J=6.4 Hz, 3H).

¹³C-NMR: 150.25, 148.28, 145.86, 144.74, 135.57, 133.69, 132.40, 126.83, 126.78, 124.67, 109.05, 108.87, 101.35, 63.93, 40.10, 22.00, 20.72 ppm.

Acylation of the products of Examples 31 and 32 is shown particularly in Examples 25, 24, 26 and 27, the reduction of these Examples can be accomplished according to Examples 4, 6, 8 and 10.

### D. Resolution and Selective Acylation of the Dihydro-2,3-Benzodiazepine Compounds Having 4-Amino-3-Methylphenyl Substituent

### Example 33

### (±)-5-(4-Amino-3-methylphenyl)-8-methyl-8,9-dihydro-7H-1,3-dioxolo[4,5-h] [2,3] benzodiazepine (VIII)

To the solution of 3.39 g (10.0 mmoles) of (±)-8-Methyl-5-(3-methyl-4-nitrophenyl)-8,9-dihydro-7H-1,3-dioxolo[4,5-h] [2,3]benzodiazepine in a mixture of 50 ml of methanol and 100 ml of dichloromethane, about 3.0 μg of wet Raney-nickel catalyst are added, then under vigorous stirring 1.7 ml (35.0 mmoles) of 98% hydrazine hydrate are added.

Following 45 minutes of an additional stirring period the catalyst is filtered off, washed with dichloromethane, the filtrate is evaporated and the residue is triturated with 50 ml of water and solidified. The raw product is recrystallised from acetonitrile.

Thus, the yield of the title product is 2.41 g (78%) of orange crystals.

Melting point: 97-100° C.

IR (KBr): 3483, 3344, 2626, 1574, 1316, 1033 cm⁻¹.

¹H-NMR (CDCl₃, mp 200): 7.60 (s, 1H), 7.52 (d, J=8.8 Hz, 1H), 6.88 (s, 1H), 6.76 (s, 1H), 6.72 (d, J=8.8 Hz, 1H), 6.09 (d, J=1.1 Hz, 1H), 6.08 (d, 1H), 4.32 (bm, 3H), 2.93 (dd, J1=5.9 Hz, J2=14.3 Hz, 1H), 2.47 (dd, J1=4.0 Hz, J2=14.7 Hz, 1H), 2.20 (s, 1H), 1.29 (d, J=6.2 Hz, 3H) ppm.

### Example 34

### (S)-(−)-5-(4-Amino-3-methylphenyl)-8-methyl-8,9-dihydro-7H-1,3-dioxolo[4,5-h] [2,3] benzodiazepine (VIII/A)

### Step a.)

In 144 ml of anhydrous ethanol, 3.09 g (10.0 mmoles) of (±)-5-(4-Amino-3-methylphenyl)-8-methyl-8,9-dihydro-7H-1,3-dioxolo[4,5h][2,3]benzodiazepine and 0.98 g (4.0 mmoles) of L-tartaric acid semi-4-chloroanilide are dissolved at the boiling point of the solvent. Then the salt is crystallised for 20 hours at room temperature. The precipitated crystals are washed with anhydrous ethanol, then dried. The obtained 1.4 g of salt is recrystallised from 98 ml of anhydrous ethanol. Thus, the yield of the title product is 1.04 g (36%).

Melting point: 193-196° C.

[α]²⁰_{D}=−22.12° (c=1, MeOH)

IR (KBr): 3440, 3347, 3260, 1675, 1647, 1597, 1036 cm⁻¹.

¹H-NMR (DMSO-d₆): 9.77 (bs, 1H), 7.77 (d, J=8.9 Hz, 2H), 7.36 (d, J=8.9 Hz, 2H), 7.13 (s, 1H), 7.03 (dd, J1=1.6 Hz, J2=8.3 Hz, 1H), 6.90 (s, 1H), 6.76 (d, J=8.3 Hz), 6.48 (s, 1H), 6.03 (s, 2H), 4.39 (m, 2H), 3.89 (hz, J=5.0 Hz, 1H), 2.67 (dd, J1=6.1 Hz, J2=13.6 Hz, 1H), 2.32 (dd, J1=4.3 Hz, J2=13.6 Hz, 1H), 2.04 (s, 3H), 1.06 (d, J=6.3 Hz, 3H) ppm.

¹³C-NMR (DMSO-d₆): 173.70, 170.92, 159.80, 147.83, 147.28, 145.16, 137.63, 134.76, 130.10, 128.65, 127.23, 127.16, 126.24, 121.27, 120.38, 113.30, 108.80, 108.44, 101.20, 73.73, 72.03, 62.76, 20.76, 17.70 ppm.

### Step b.)

A suspension of 1.04 g of salt prepared in step a.) in 20 ml of chloroform is mixed with 20 ml of saturated aqueous sodium hydrogen carbonate solution, then the mixture is agitated until clear phases are formed. The organic layer is washed with 3×20 ml of water, dried over sodium sulphate, then evaporated. The obtained product can be used without further purification.

### Example 35

### (R)-(+)-5-(4-Amino-3-methylphenyl)-8-methyl-8,9-dihydro-7H-1,3-dioxolo[4,5-h] [2,3] benzodiazepine (VIII/A)

### Step a.)

In 144 ml of boiling anhydrous ethanol, 3.09 g (10.0 mmoles) of (±)-5-(4-Amino-3-methylphenyl)-8-methyl-8,9-dihydro-7H-1,3-dioxolo[4,5h][2,3]benzodiazepine and 0.98 g (4.0 mmoles) of D-tartaric acid semi-4-chloroanilide are dissolved. Then the salt is crystallised for 20 hours at room temperature. The precipitated crystals are washed with anhydrous ethanol, then dried. The yield is 1.43 g product which is recrystallized from 98 ml of anhydrous ethanol.

Thus, the yield is 1.06 g (37%) of the title product.

The melting point is 193-196° C.

[α]²⁰_{D}=+22.41° (c=1, MeOH)

IR (KBr): 3440, 3347, 3260, 1675, 1647, 1597, 1036 cm⁻¹.

¹H-NMR (DMSO-d₆): 9.77 (bs, 1H), 7.77 (d, J=8.9 Hz, 2H), 7.36 (d, J=8.9 Hz, 2H), 7.13 (s, 1H), 7.03 (dd, J1=1.6 Hz, J2=8.3 Hz, 1H), 6.90 (s, 1H), 6.76 (d, J=8.3 Hz), 6.48 (s, 1H), 6.03 (s, 2H), 4.39 (m, 2H), 3.89 (hz, J=5.0 Hz, 1H), 2.67 (dd, J1=6.1 Hz, J2=13.6 Hz, 1H), 2.32 (dd, J1=4.3 Hz, J2=13.6 Hz, 1H), 2.04 (s, 3H), 1.06 (d, J=6.3 Hz, 3H).

¹³C-NMR (DMSO-d₆): 173.70, 170.92, 159.80, 147.83, 147.28, 145.16, 137.63, 134.76, 130.10, 128.65, 127.23, 127.16, 126.24, 121.27, 120.38, 113.30, 108.80, 108.44, 101.20, 73.73, 72.03, 62.76, 20.76, 17.70.

### Step b.)

A suspension of 1.06 g of salt prepared in step a.) in 20 ml of chloroform is mixed with 20 ml of saturated sodium hydrogen carbonate solution, then the mixture is agitated until clear phases are formed. The organic layer is washed with 3×20 ml of water, dried with sodium sulphate, then evaporated. The obtained product can be used without further purification.

### Example 36

### (S)-(+)-7-Acetyl-5-(4-amino-3-methylphenyl)-8-methyl-8,9-dihydro-7H-1,3-dioxolo[4,5-h] [2,3] benzodiazepine (I/S)

To a solution of 3.09 g (10.0 mmoles) of (S)-(−)-5-(4-Amino-3-methylphenyl)-8-methyl-8,9-dihydro-7H-1,3-dioxolo[4,5-h][2,3]benzodiazepine in 10 ml of chloroform, 1.39 ml (10.0 mmoles) of triethylamine are added. Then the reaction mixture is cooled to −10° C. and 0.94 ml (10.0 mmoles) of acetic acid anhydride is added and stirred for 1.5 hours. Then the reaction mixture is added to 70 ml of saturated sodium hydrogen carbonate solution under stirring, the organic layer is separated, washed with 5×15 ml of water, dried oversodium sulphate and evaporated. The raw product is recrystallized from anhydrous ethanol.

The yield is 2.12 g (60%) of pale yellow product.

The melting point is 119-122 C.

[α]²⁰_{D}=+478.1° (c=1, CHCl₃)

IR (KBr): 3484, 1658, 1342, 1039 cm⁻¹.

¹H-NMR (CDCl₃): 7.47 (d, J=1.3 Hz, 1H), 7.32 (dd, J1=2.1 Hz, J2=8.3 Hz, 1H), 6.77 (s, 1H), 6.65 (d, J=8.2 Hz, 1H), 6.58 (s, 1H), 6.01 (d, J=1.4 Hz, 1H), 5.97 (d, J=1.4 Hz, 1H), 5.21 (m, 1H), 3.99 (bs, 2H), 2.66 (m, 2H), 2.19 (s, 3H), 2.01 (s, 3H), 1.31 (d, J=6.3 Hz, 3H) ppm.

¹³C-NMR: 173.91, 168.63, 149.02, 147.94, 146.08, 135.19, 131.61, 129.25, 127.24, 125.97, 121.61, 113.97, 109.28, 108.63, 101.44, 61.21, 38.77, 22.53, 18.20, 17.29 ppm.

### Example 37

### (R)-(−)-7-Acetyl-5-(4-amino-3-methylphenyl)-8-methyl-8,9-dihydro-7H-1,3-dioxolo[4,5-h] [2,3] benzodiazepine (I/R)

To a solution of 3.09 g (10.0 mmoles) of (R)-(+)-5-(4-Amino-3-methylphenyl)-8-methyl-8,9-dihydro-7H-1,3-dioxolo[4,5-h][2,3]benzodiazepine in 10 ml of chloroform, 1.39 ml (10.0 mmoles) of triethylamine are added. Then the reaction mixture is cooled to −10° C. and 0.94 ml (10.0 mmoles) of acetic acid anhydride is added and stirred for 1.5 hours. Then the reaction mixture is added to 70 ml of saturated sodium hydrogen carbonate solution under stirring, the organic layer is separated, washed with 5×15 ml of water, dried over sodium sulphate and evaporated. The raw product is recrystallized from anhydrous ethanol.

The yield is 2.14 g (61%) of pale yellow product.

The melting point is 121-124° C.

[α]²⁰_{D}=−479.3° (c=1, CHCl₃)

IR (KBr): 3484, 1658, 1342, 1039 cm⁻¹.

¹H-NMR (CDCl₃): 7.47 (d, J=1.3 Hz, 1H), 7.32 (dd, J1=2.1 Hz, J2=8.3 Hz, 1H), 6.77 (s, 1H), 6.65 (d, J=8.2 Hz, 1H), 6.58 (s, 1H), 6.01 (d, J=1.4 Hz, 1H), 5.97 (d, J=1.4 Hz, 1H), 5.21 (m, 1H), 3.99 (bs, 2H), 2.66 (m, 2H), 2.19 (s, 3H), 2.01 (s, 3H), 1.31 (d, J=6.3 Hz, 3H) ppm.

¹³C-NMR: 173.91, 168.63, 149.02, 147.94, 146.08, 135.19, 131.61, 129.25, 127.24, 125.97, 121.61, 113.97, 109.28, 108.63, 101.44, 61.21, 38.77, 22.53, 18.20, 17.29 ppm.

### Example 38

### (S)-(+)-5-(4-amino-3-methylphenyl)-8-methyl-7-propionyl-8,9-dihydro-7H-1,3-dioxolo[4,5-h] [2,3]benzodiazepine (I/S)

To a solution of 3.09 g (10.0 mmoles) of (S)-(−)-5-(4-Amino-3-methylphenyl)-8-methyl-8,9-dihydro-7H-1,3-dioxolo[4,5-h][2,3]benzodiazepine in 10 ml of chloroform, 1.39 ml (10.0 mmoles) of triethylamine are added. Then the reaction mixture is cooled to −10° C. and 1.28 ml (10.0 mmoles) of propionic acid anhydride are added and stirred for 1.5 hours. Then the reaction mixture is added to 70 ml of saturated sodium hydrogen carbonate solution under stirring, the organic layer is separated, washed with 5×15 ml of water, dried over sodium sulphate and evaporated. The raw product is recrystallized from anhydrous ethanol.

The yield is 2.64 g (64%) of pale yellow product.

The melting point is 176-178° C.

[α]²⁰_{D}=+433.7° (c=1, CHCl₃)

IR (KBr): 3355, 3245, 1631, 1038 cm⁻¹.

¹H-NMR (CDCl₃, i400): 7.46 (bs, 1H), 7.33 (dd, J1=1.8 Hz, J2=8.2 Hz, 1H), 6.76 (s, 1H), 6.66 (d, J=8.3 Hz, 1H), 6.57 (s, 1H), 6.00 (d, J=1.3 Hz, 1H), 5.95 (d, J=1.3 Hz, 1H), 5.21 (m, 1H), 4.05 (b, 1H), 2.65 (m, 2H), 2.47 (m, 1H), 1.19 (m, 1H), 2.19 (s, 3H), 1.30 (d, J=6.4 Hz, 3H), 1.03 (t, J=7.5 Hz, 3H) ppm.

### Example 39

### (R)-(−)-5-(4-amino-3-methylphenyl)-8-methyl-7-propionyl-8,9-dihydro-7H-1,3-dioxolo[4,5-h] [2,3]benzodiazepine (I/R)

To a solution of 3.09 g (10.0 mmoles) of (R)-(+)-5-(4-Amino-3-methylphenyl)-8-methyl-8,9-dihydro-7H-1,3-dioxolo[4,5-h][2,3]benzodiazepine in 10 ml of chloroform, 1.39 ml (10.0 mmoles) of triethylamine are added. Then the reaction mixture is cooled to −10° C. and 1.28 ml (10.0 mmoles) of propionic acid anhydride are added and stirred for 1.5 hours. Then the reaction mixture is added to 70 ml of saturated sodium hydrogen carbonate solution under stirring, the organic layer is separated, washed with 5×15 ml of water, dried over sodium sulphate and evaporated. The raw product is recrystallized from anhydrous ethanol.

The yield is 2.58 g (60%) of pale yellow product.

The melting point is 175-178° C.

[α]²⁰_{D}=−415.4° (c=1, CHCl₃)

IR (KBr): 3355, 3245, 1631, 1038 cm⁻¹.

¹H-NMR (CDCl₃, i400): 7.46 (bs, 1H), 7.33 (dd, J1=1.8 Hz, J2=8.2 Hz, 1H), 6.76 (s, 1H), 6.66 (d, J=8.3 Hz, 1H), 6.57 (s, 1H), 6.00 (d, J=1.3 Hz, 1H), 5.95 (d, J=1.3 Hz, 1H), 5.21 (m, 1H), 4.05 (b, 1H), 2.65 (m, 2H), 2.47 (m, 1H), 1.19 (m, 1H), 2.19 (s, 3H), 1.30 (d, J=6.4 Hz, 3H), 1.03 (t, J=7.5 Hz, 3H) ppm.

1-36. (canceled)

37. An enantiomeric compound according to the formula I 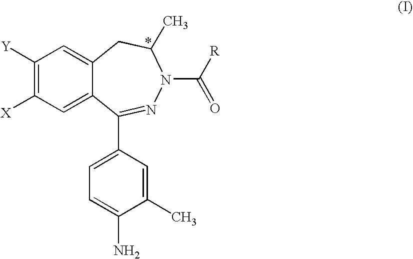
wherein
the configuration of the chiral carbon atom is R,
X stands for hydrogen, a halogen or an alkoxy group,
Y stands for hydrogen or a halogen, or
X and Y together may stand for a methylenedioxy group, with the proviso that if X stands for chloro, Y is hydrogen,
R stands for a methyl or ethyl group and pharmaceutically acceptable acid additional salts thereof.

38. An enantiomeric compound according to the formula V 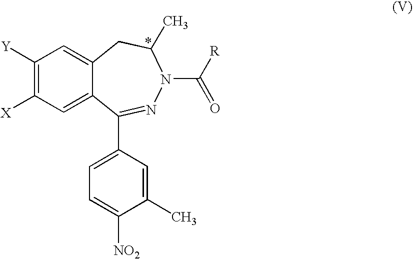
wherein the configuration of the chiral carbon atom is R or S,
X is hydrogen, halo or alkoxy,
Y is hydrogen or halo, or
X and Y together form a methylenedioxy group, with the proviso that if X stands for chloro, Y is hydrogen, and
R is methyl or ethyl.

39. An enantiomeric compound of the Formula (XII) 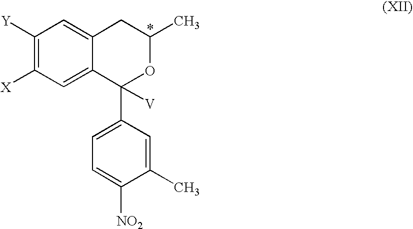
wherein
the configuration of the chiral carbon atom is R or S,
X stands for a hydrogen atom, halogen atom or an alkoxy group,
Y stands for a hydrogen or halogen, or
X and Y together may stand for a methylenedioxy group, with the proviso that if X stands for chloro, Y is hydrogen, and
V stands for a hydrogen atom or a hydroxyl group.

40. A compound of the Formula XIV, 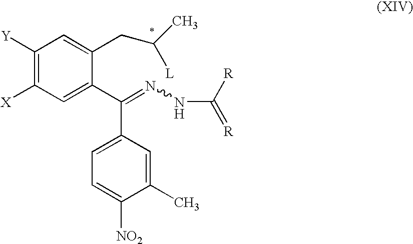
wherein
the compounds are mixtures of E and Z isomers, the configuration of the chiral carbon atom is R or S,
X stands for a hydrogen atom, halogen atom or an alkoxy group,
Y stands for a hydrogen or halogen, or
X and Y together may stand for a methylenedioxy group, with the proviso that if X stands for chloro, Y is hydrogen,
R is methyl or ethyl.
L stands for a hydroxyl group or an alkyl- or arylsulphonyl group.

41. A racemic or enantiomeric compound of the Formula (III) 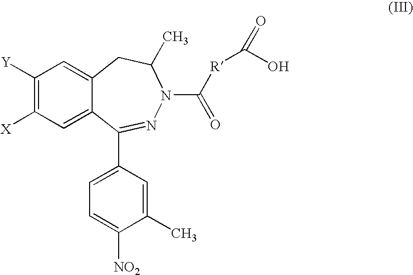
wherein
X and Y together stand for a methylenedioxy group,
R′ stands for a substituted arylene, alkylene or alkenylene, or an addition salt with chiral bases thereof.

42. A racemic or enantiomeric compound of the Formula (VI) 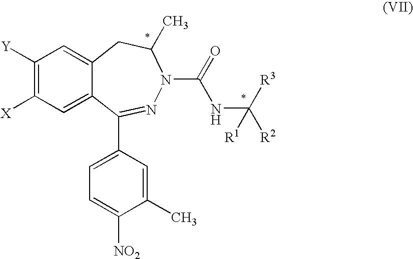
wherein
X and Y together stand for a methylenedioxy group,

43. A diastereomeric compound having high stereo chemical purity according to the formula (VII) 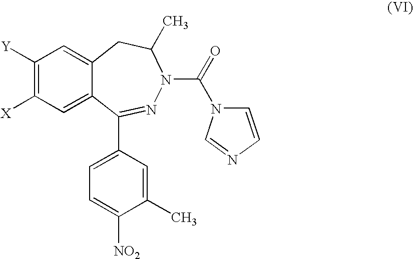
wherein
X and Y together stand for a methylenedioxy group,
R¹, R², R³ are different and stand for a hydrogen atom, substituted, or unsubstituted aliphatic or branched saturated or unsaturated alkyl, substituted or unsubstituted aryl or aralkyl group.

44. A racemic compound according to the formula VIII 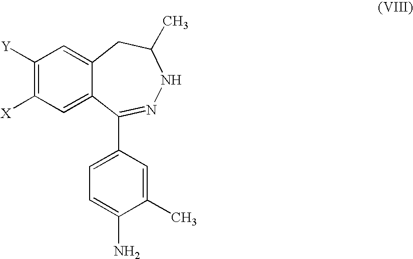
wherein
X and Y together stand for a methylenedioxy group, or an acid addition salt with optically active acids thereof.

45. An enantiomeric compound according to the formula VIII/A 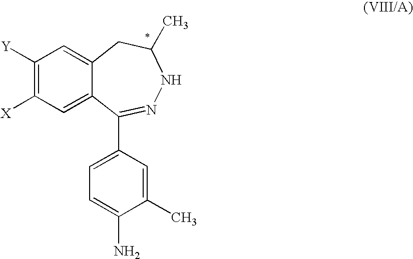
wherein
the configuration of the chiral carbon atom is R or S,
X and Y together stand for a methylenedioxy group, or an acid addition salts with optically active acids thereof.

46. A compound of the Formula (I) defined in claim 37 selected from the group consisting of:
(R)-(−)- and (S)-(+)-5-(4-amino-3-methylphenyl)-8-methyl-7-propionyl-8,9-dihydro-7H-1,3-dioxolo[4,5-h][IR,IS][2,3] benzodiazepine[1R,1S],
(R)-(−)-, and (S)-(+)-7-acetyl-5-(4-amino-3-methylphenyl-8-methyl-8,9-dihydro-7H-1,3-dioxolo[4,5-h][2,3] benzodiazepine[1R,1S], and
(S)-(+)- and (R)-(−)-3-acetyl-1-(4-amino-3-methylphenyl)-8-chloro-4-methyl-4,5-dihydro-3H-2,3-benzodiazepine[1R,1S] and pharmaceutically acceptable acid addition salts thereof.

47. A compound of the Formula (V) defined in claim 38, selected from the group consisting of
(R)-(−)-, and (S)-(+)-8-methyl-5-(3-methyl-4-nitrophenyl)-8,9-dihydro-7H-1,3-dioxolo[4,5-H][2,3]benzodiazepine,
(R)-(−)-, and (S)-(+)-7-acetyl-8-methyl-5-(3-methyl-4-nitrophenyl)-8,9-dihydro-7H-1,3-dioxolo[4,5-H][2,3]benzodiazepine and
(S)-(−)- and (R)-(+)-3-acetyl-8-chloro-4-methyl-1-(3-methyl-4-nitrophenyl-4,5-dihydro-3H-2,3-benzodiazepine.

48. A compound of the Formula XII defined in claim 39 selected from the group consisting of (5RS,7S)-, and (5RS,7R)-7-Methyl-5-(3-methyl-4-nitrophenyl)-7,8-dihydro-5H-1,3-dioxolo[4,5 glisochromane, and (1RS.3R) and (1RS,3S-7-Chloro-3-methyl-1-(3-methyl-4-nitrophenyl)-isochromane.

49. A compound of the formula (XII) defined in claim 39 and selected from the group consisting of (5RS,7R)-, and (5RS,7S)-7-methyl-5-(3-methyl-4-nitrophenyl-7,8-dihydro-5H-1,3 dioxolo[4,5-g]isochroman-5-ol, and (1RS.3R)- and (1RS,3S)-7-Chloro-3-methyl-1-(3-methyl-4-nitrophenyl-isochroman-1-ol.

50. A compound of the Formula (XIV) defined in claim 40 and selected from the group consisting of (R)-, and (S)-Acetic acid-[[6-(2-hydroxypropyl)-1,3-benzodioxol-5-yl](3-methyl-4-nitrophenyl-methylene]hydrazide, (R)-, and (S)-Propionic acid-[[6-(2-hydroxypropyl)-1,3-benzodioxol-5-yl](3-methyl-4-nitrophenyl)-methylene]hydrazide, and (R)- and (S)-acetic acid[[5-chloro-2-(2-hydroxypropyl)-phenyl]-(3-methyl-4-nitrophenyl)methylene]-hydrazide.

51. A compound of the Formula (XIV) defined in claim 40 and selected from the group consisting of:
(R)-, and (S)-Acetic acid-[[6-[2-[(methylsulphonyl-oxy]-propyl)-1,3-benzodioxol-5-yl](3-methyl-4-nitrophenyl)-methylene]hydrazide,
(R)- and (S)-Propionic acid-[[6-[2-[(methylsulphonyl)-oxy]-propyl)-1,3-benzodioxol-5-yl](3-methyl-4-nitrophenyl)-methylene]hydrazide, and
(S) and (R) acetic acid [[2-[2-[(methylsulphonyl)-1-oxy]-propyl-5-chlorophenyl](3-methyl-4-nitrophenyl-methylene]hydrazide.

52. (S)-(−)- and (R)-(+)-8-Methyl-5-(3-methyl-4-nitrophenyl)-8,9-dihydro-7H-1,3-dioxolo[4,5-h][2,3]benzodiazepine.

53. (±)-, (S)-(+)- and (R)-(−)-4-(8-Methyl-5-(3-methyl-4-nitrophenyl-8,9-dihydro-7H-1,3-dioxolo[4,5-h][2,3] benzodiazepine-7-yl)-4-oxo-but-2-en carboxylic acid and addition salts with chiral bases thereof of the Formula (III) defined in claim 41.

54. The compound or Salt of the formula (III) defined in claim 41 selected from the group consisting of:
(S)-(+)-4-(8-Methyl-5-(3-methyl-4-nitrophenyl-8,9-dihydro-7H-1,3-dioxolo[4,5-h][2,3]benzodiazepine-7-yl)-4-oxo-but-2-en carboxylic acid (R)-(+)α-methyl-benzylammonium salt, and (R)-(−)-4-(8-Methyl-5-(3-methyl-4-nitrophenyl)-8,9-dihydro-7H-1,3-dioxolo[4,5-h] [2,3]benzodiazepine-7-yl-4-oxo-but-2-encarboxylic acid (S)-(−)-α-methyl-benzylammonium salt.

55. (±)-8-Methyl-5-(3-methyl-4-nitrophenyl)-8,9-dihydro-7H-1,3-dioxolo[4,5-h][2,3] benzodiazepine-7-carboxylic acid-imidazolide of the Formula (VI) defined in claim 42.

56. A compound of the Formula (VII) defined in claim 43 which is selected from the group consisting of: (+)-7-(N-(1R-phenylethyl-carbamoyl-8(R-methyl-5-(3-methyl-4-nitrophenyl-8,9-dihydro-7H-1,3-dioxolo[4,5-hj[2,3]benzodiazepine, and (−)-7-(N-(1(S)-phenylethyl)-carbamoyl-8(S)methyl-5-(3-methyl-4-nitrophenyl-8,9-dihydro-7H-1,3-dioxolo[4,5-h][2,3]benzodiazepine.

57. (±)-, (S)-(−)- and (R)-(−)-5-(4-Amino-3-methylphenyl-8-methyl-8,9-dihydro-7H-1,3-dioxolo[4,5-h][2,3] benzodiazepine and salts composed with optically active carboxylic acids thereof.

58. Pharmaceutical composition comprising an enantiomeric dihydro-2,3-benzodiazepine compound according to the formula (I), defined in claim 37 wherein the configuration of the chiral carbon atom is R,
X stands for a hydrogen atom, halogen atom or alkoxy group,
Y stands for a hydrogen or halogen atom, or
X and Y together may stand for a methylenedioxy group, with the proviso that if X stands for chloro, Y is hydrogen,
R stands for a methyl or ethyl group, or pharmaceutically acceptable acid addition salts thereof and the usual carrier(s).

59. Pharmaceutical composition according to claim 58 comprising as active ingredient (R)-(−)-5-(4-amino-3-methylphenyl)-8-methyl-7-propionyl-8,9-dihydro-7H-1,3-dioxolo[4,5-h][2,3]benzodiazepine, or (R)-(−)-7-acetyl-5-(4-amino-3-methylphenyl)-8-methyl-8,9-dihydro-7H-1,3-dioxolo[4,5-h][2,3]benzodiazepine, or (R)-(−)-3-acetyl-1-(4-amino-3-methylphenyl)-8-chloro-4-methyl-4,5-dihydro-3H-2,3-benzodiazepine or pharmaceutically acceptable acid addition salts thereof.

60. Pharmaceutical composition according to claim 58 containing 0.1-95 weight % of the active ingredient.

61. Pharmaceutical composition according to claim 58 comprising a composition suitable for oral, parenteral, rectal, transdermal or topical use.

62. A process for the preparation of a compound of high enantiomeric purity of the formula I as defined in claim 37 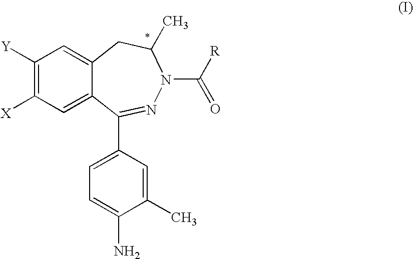
which comprises the step of reducing the nitro group of the corresponding dihydro-2,3-benzodiazepine compound according to the formula (V) 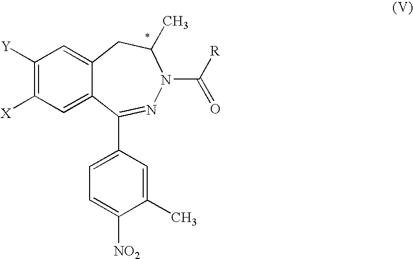
and optionally transforming the reduced product into a pharmaceutically acceptable acid addition salt thereof.

63. A process for preparing the compound of the Formula (I) according to claim 37, which comprises the steps of
(a) reacting the phenyl-propanol-2 derivative according to the formula (X) 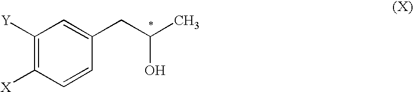
of high enantiomeric purity, with a 4-nitrobenzaldehyde according to the formula (XI) 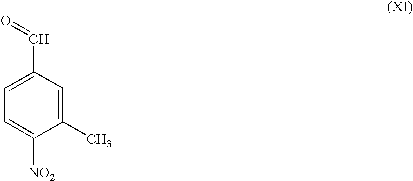
thereafter
(b) oxidizing the obtained benzo[b]pyrane according to the formula (XII) 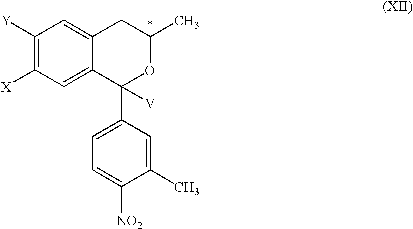
which is a mixture of isomers and wherein V stands for a hydrogen atom, to a hemiketal-type compound according to the formula (XIII) 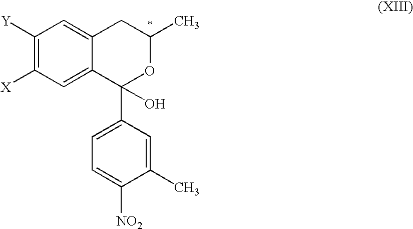
c) reacting the thus obtained diastereomeric hemiketal type derivatives of the formula (XIII) 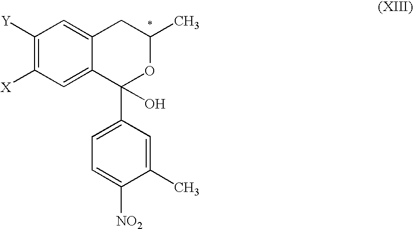
with an aliphatic carboxylic acid hydrazide, preferably with acetic acid hydrazide,
(d) reacting further the obtained hydrazone according to the formula (XIV), 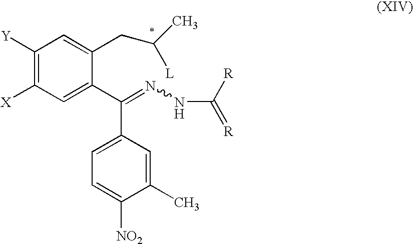
which is a mixture of E and Z isomers and wherein L stands for a hydroxyl group, with an alkylsulphonyl halogenide, or an arylsulphonyl halogenide to obtain aryl or alkylsulphonylated hydrazone compounds according to the formula (XV) 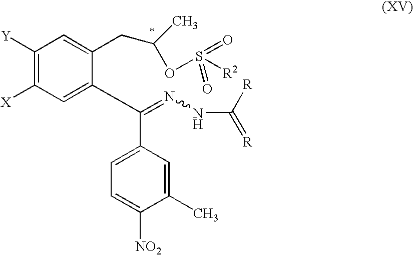
which is a mixture of E and Z isomers, wherein R2 stands for an aryl, C1-C4 alkyl group,
(e) transforming this sulphonylated hydrazon derivative in to a dihydro-2,3-benzodiazepine derivative according to the formula (V) 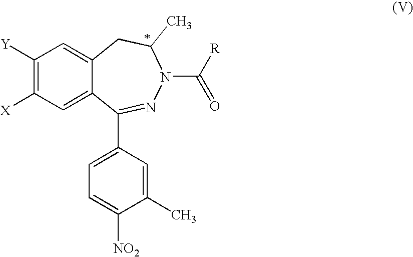
of high enantiomeric purity through an intramolecular cyclisation reaction wherein R is a C₁ to C₄ alkyl group, and
(f) finally reducing the obtained product to a dihydro-2,3-benzodiazepine derivative according to the formula (I), and optionally forming a pharmaceutical acceptable acid addition salt thereof.

64. A process for the preparation of (R)-(−)-7-acetyl-5-(4-amino-3-methylphenyl)-8-methyl-8,9-dihydro-7H-1,3-dioxolo[4,5-h][2,3]benzodiazepine as defined in claim 46 of high enantiomeric purity and pharmaceutically acceptable acid addition salts thereof, which comprises the steps of
(a) reacting (S)-α-methyl-1,3-benzodioxol-5-ol of high enantiomeric purity with 3-methyl-4-nitrobenzaldehyde to obtain a compound of the Formula (XII) 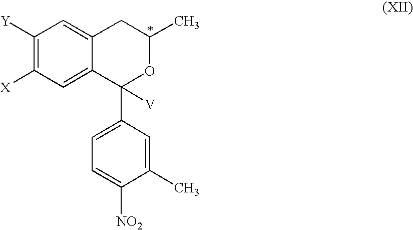
(b) oxidizing the obtained compound of the formula (XII), which is a mixture of diastereomers, wherein X and Y together stand for a methylenedioxy group, and V stands for a hydrogen atom, to form a hemiketal according to the formula (XIII), 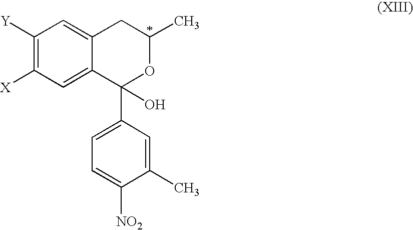
wherein X and Y together stand for a methylenedioxy group, and V stands for a hydroxyl group,
(c) reacting the thus obtained hemiketal with acetic acid hydrazide to obtain a compound of the Formula (XIV), 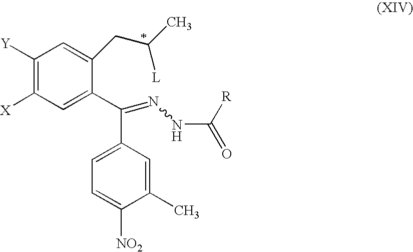
which is a mixture of E and Z isomers, wherein X and Y together stand for a methylenedioxy group, L stands for a hydroxyl group, with an alkylsulphonyl halogenide, or an arylsulphonyl halogenide,
(d) reacting the obtained compound according to the formula (XIV) to form an aryl- or alkylsulphonylized hydrazone according to the formula (XV) 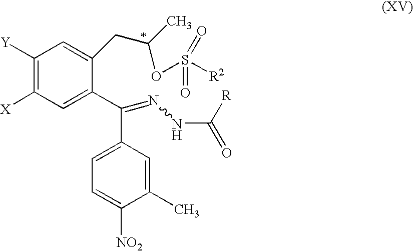
which is a mixture of E and Z isomers, wherein X and Y together stand for a methylenedioxy group, L stands for an alkylsulphonyloxy or arylsulphonyloxy group, and
(e) transforming this product by an intramolecular cyclisation reaction into a dihydro-benzodiazepine according to the formula (V) 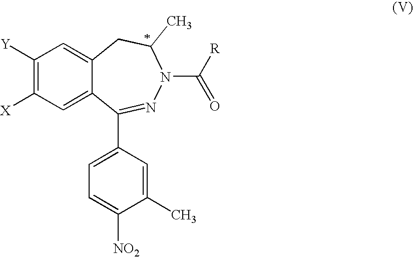
of high enantiomeric purity, wherein X and Y together stand for a methylenedioxy group, thereafter reducing the nitro group to an amino group and, if desired, transforming the obtained product into a pharmaceutically acceptable acid addition salt thereof.

65. A process for the preparation of (R)-(−)-3-acetyl-1-(4-amino-3-methylphenyl)-8-chloro-4-methyl-4,5-dihydro-3H-2,3-benzodiazepine as defined in claim 46 of high enantiomeric purity, which comprises the steps
(a) reacting the (S)-1-(4-chlorophenyl)-propanol-2 of high enantiomeric purity with 3-methyl-4-nitrobenzaldehyde, to obtain a benzo[b]pyrane compound according to the formula (XII), 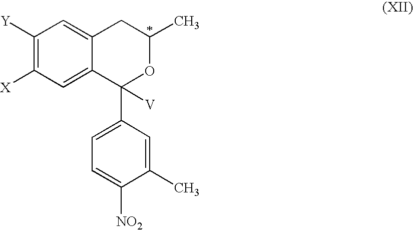
which is a diastereomer mixture, wherein X stands for a chloro atom, Y and V stand for hydrogen atoms,
(b) oxidizing the benzo[b]pyrane compound to a hemiketal compound according to the formula (XIII), 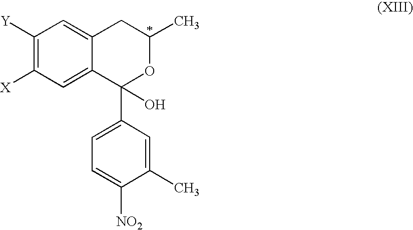
wherein X stands for a chloro atom, Y stands for a hydrogen atom and V stands for a hydroxyl group,
(c) reacting the thus obtained product with acetic acid hydrazide to form a hydrazone compound according to the formula (XIV) 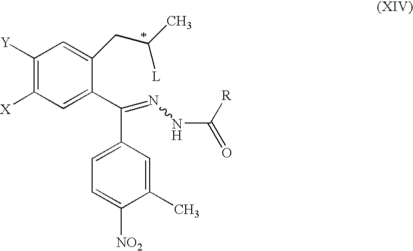
which is a mixture of E and Z isomers, wherein X stands for a chloro atom, Y stands for a hydrogen atom and L stands for a hydroxyl group,
(d) reacting this hydrazone compound with an alkylsulphonyl or arylsulphonyl compound to obtain a compound of the Formula (XV) 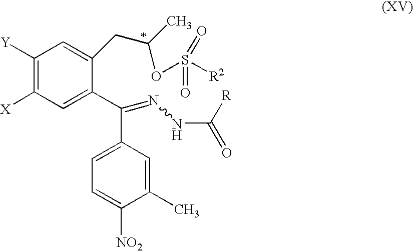
(e) further reacting the obtained aryl- or alkylsulfornylated hydrazone compound of the Formula (XV) by means of an intramolecularcyclization reaction, to form a dihydrobenzodiazepine compound of the Formula (V) 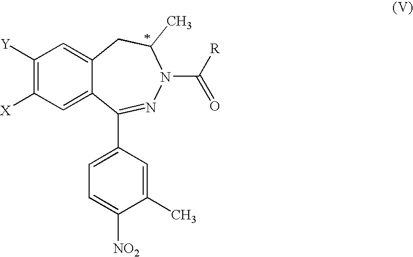
and
(f) reducing the compound of the Formula (V) to obtain the compound of the Formula (I)

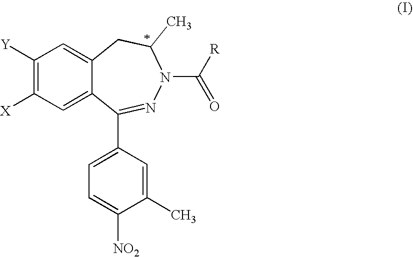

66. A process for the preparation of dihydro-2,3-benzodiazepine compound having high diastereomeric purity according to the formula (I) as defined in claim 37 which comprises the steps
(a) acylating racemic dihydro-2,3-benzodiazepine compound according to the formula (II) 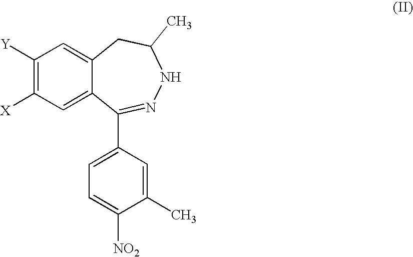
with an aliphatic or aromatic dicarboxylic acid to obtain a half acid-half amide compound according to the formula (III) 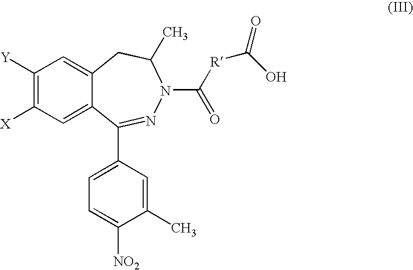
wherein R′ stands for a substituted arylene, alkylene or alkenylene,
(b) reacting the compound of formula (III) with a single enantiomer of a chiral base to obtain a pair of diastereomeric salts according to the formula (IV) 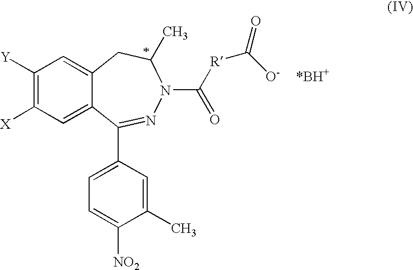
wherein “BH” is a protonated form of a single enantiomer of a chiral amine, Separating the enantiomer pairs into individual enantiomers, obtaining thus a diastereomer salt in the desired enantiomer form, thereafter
(c) releasing from the diastereomer salt the half acid-half amide compound according to the formula (III/A) 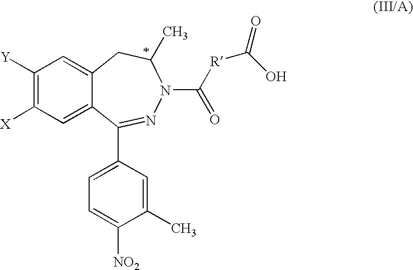
wherein R′ stands for a substituted or arylene, alkylene or alkenylene,
(d) then hydrolyzing this compound to obtain the half acid-half amide compound of the formula (II/A) 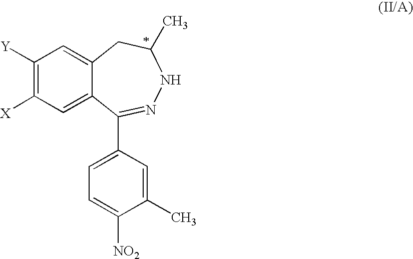
(e) acylating the compound according to formula (II/A) using aliphatic carboxylic derivatives to obtain a compound of the Formula (V) 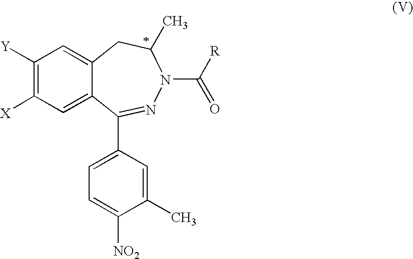
, and
(f) thereafter reducing the nitro group of the obtained compound of the Formula (V) to form a dihydro-2,3-benzodiazepine compound according to the formula (I) of high enantiomeric purity and optionally transforming this compound into a pharmaceutically acceptable acid addition salt thereof.

67. A process for the preparation of a dihydro-2,3-benzodiazepine derivatives of the formula (I) of high enantiomeric purity as defined in claim 37 wherein X and Y together form a methylenedioxy group, which comprises the steps of
(a) reacting a racemic dihydro-2,3-benzodiazepine according to the formula (II) 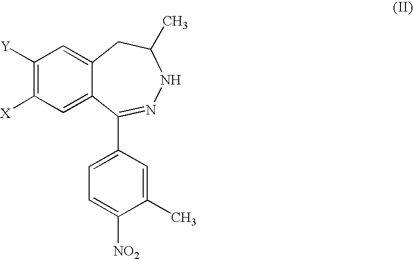
with 1,1′-carbonyl-diimidazole, then
(b) reacting the obtained racemic carbonyl-diimidazole according to the formula (IV) 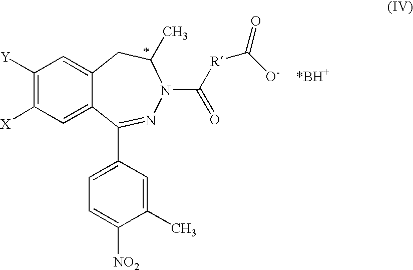
wherein R′ stands for a substituted arylene, alkylene, or alkenylene with an enantiomer of a chiral base, then
(c) separating the components of the obtained diastereomer mixture of the dihydro-2,3-benzodiazepine according to the formula (VII) 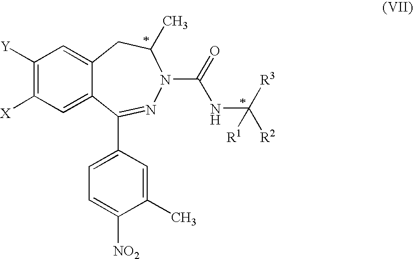
wherein the configuration of the chiral carbon atom of the Dihydro-2,3-benzodiazepine is R or S and the configuration of the other chiral carbon atom depends on the used chiral amine, X and Y are together methylenedioxy group, R1, R2, R3 are different from each other and stand for a hydrogen atom, substituted or unsubstituted, aliphatic or branched, saturated or unsaturated alkyl, substituted or unsubstituted aryl or aralkyl group followed by a recrystallization step if required,
(d) then hydrolyzing this compound under acidic conditions to obtain an enantiomeric dihydro-2,3-benzodiazepine derivatives according to the formula (II/A) 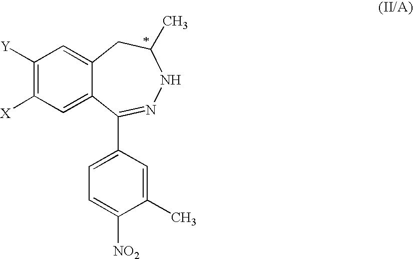
(e) acylating the compound of the Formula (II/A)
with an aliphatic carboxylic acid derivative, to obtain a compound of the Formula (V) 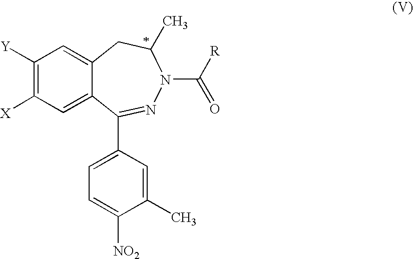
and finally
(f) reducing the nitro group of the obtained compound of the Formula (V) to obtain a dihydro-2,3-benzodiazepine derivative according to the formula (I) wherein X and Y together form a ethylenedioxy group of high enantiomeric purity and optionally transforming them into a pharmaceutically acceptable salt thereof.

68. A process for the preparation of dihydro-2,3-benzodiazepine of high enantiomeric purity according to the formula (I), as defined in claim 37 wherein X and Y together stand for a methylenedioxy group, which comprises the steps of acylating the unsubstituted cyclic nitrogen atom at the position 3 of the corresponding enantiomeric benzodiazepine compound according to the formula (VIII/A) 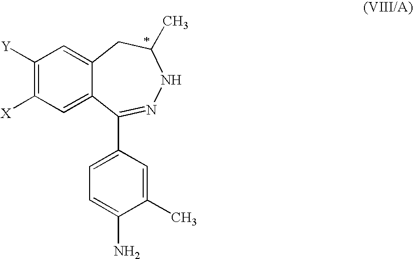
with a C₁₋₄ carboxylic acid chloride or an acid anhydride, and optionally transforming the obtained product according to the formula (I) into a pharmaceutically acceptable acid addition salt thereof.

69. A process for preparing the compound of Formula (I) as defined in claim 37 wherein X and Y together form a methylenedioxy group, which comprises the steps of
(a) reducing the racemic dihydro-2,3-benzodiazepine according to the formula (II), 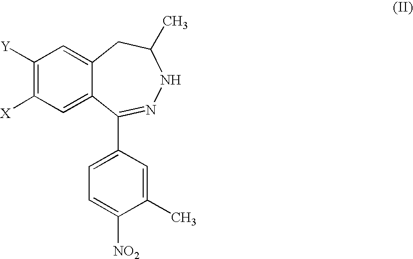
b) transforming the obtained racemic Dihydro-2,3-benzodiazepine according to the formula (VIII) 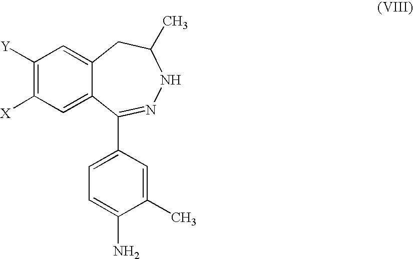
into a diastereomer acid salt pair with a single stereoisomer of a chiral carboxylic acid,
(c) separating the components of the diastereomer salts, thereafter optionally recrystallizing the diastereomer salt of the formula (IX) 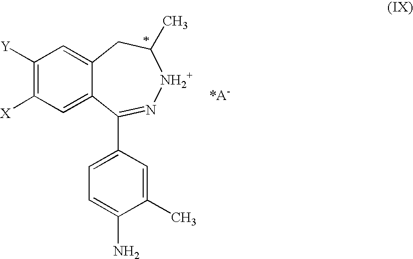
wherein *A” stands for an anion of an optically active carboxylic acid and the configuration of the chiral carbon atom of the benzo-diazepine group is R or S1 thereafter
(d) releasing from the enantiomeric dihydro-2,3-benzodiazepine containing salt the enantiomeric dihydro-2,3-benzodiazepine base according to the formula (VIII/A) 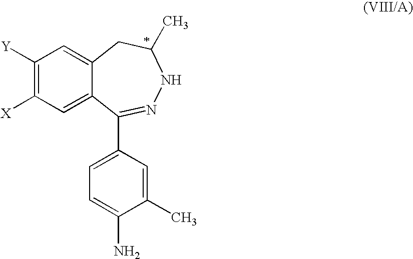
and
(e) acylating the unsubstituted cyclic nitrogen atom at the position 3 of the corresponding enantiomer benzodiazepine base according to the formula (Vlll/A), with a C₁₋₄ carboxylic acid chloride or acid anhydride, finally optionally transforming the obtained product according to the formula (I) into a pharmaceutically acceptable acid addition salt thereof.

70. A method of treating epilepsy, muscle-spasticity or stroke in a patient instead of said treatment which comprises the step of administering to said patient, a therapeutically effective amount of the compound of the Formula (I) defined in claim 37, or a pharmaceutically acceptable salt thereof.
